(19) United States
(12) Patent Application Publication
(10) Pub. No.: US 2025/0326712 A1
(43) Pub. Date: Oct. 23, 2025

(54) IONIZABLE LIPIDS

(71) Applicant: eTheRNA immunotherapies NV, Niel (BE)

(72) Inventors: Stefaan DE KOKER, Niel (BE); Sabah KASMI, Niel (BE); Ruben DE COEN, Niel (BE)

(73) Assignee: eTheRNA immunotherapies NV, Niel (BE)

(21) Appl. No.: 18/866,766

(22) PCT Filed: May 19, 2023
(86) PCT No.: PCT/EP2023/063465

§ 371 Date: Nov. 18, 2024

(30) Foreign Application Priority Data
May 20, 2022 (EP) 22174523.5

(51) Int. Cl. C07C 323/60; A61K 9/51; A61K 48/00; C07D 295/145; C07D 295/15

(52) U.S. Cl. CPC C07C 323/60; A61K 9/5123; A61K 48/0041; C07D 295/145; C07D 295/15

(57) ABSTRACT

The present invention generally relates to the field of ionizable (also termed cationic) lipids, and in particular provides a novel type of such lipids as represented by any of the formulae disclosed herein. The present invention further provides methods for making such lipids as well as uses thereof, in particular in the preparation of nanoparticle compositions, more in particular nanoparticle compositions comprising nucleic acids. It further provides vaccine formulations and pharmaceutical formulations comprising nanoparticle compositions based on the ionizable lipids disclosed herein.

### FIELD OF THE INVENTION

The present invention generally relates to the field of ionizable (also termed cationic) lipids, and in particular provides a novel type of such lipids as represented by any of the formulae disclosed herein. The present invention further provides methods for making such lipids as well as uses thereof, in particular in the preparation of nanoparticle compositions, more in particular nanoparticle compositions comprising nucleic acids. It further provides vaccine formulations and pharmaceutical formulations comprising nanoparticle compositions based on the ionizable lipids disclosed herein.

### BACKGROUND TO THE INVENTION

Nucleic acid-based drugs are being explored in a growing number of therapeutic areas. Nonetheless, due to their negative charge, size and instability, the targeted delivery of nucleic acids such as plasmid DNA, messenger RNA, short interfering RNA, single guide RNA and micro-RNAs to tissues and cells poses a major challenge. A plethora of nanoparticulate carrier systems has been explored to encapsulate and deliver nucleic acids. These nanoparticles need to combine efficient and stable encapsulation of the nucleic acid upon storage and in the extracellular environment, with maximum cellular uptake and efficient release of their payload from endosomes into the cytosol.

Lipid based nanoparticles are clinically used to deliver small interfering RNA and mRNA vaccines and represent the most advanced class of RNA delivery vehicles. Lipid based nanoparticles are typically composed of a cationic or ionizable lipid that can be protonated at acid pH, a helper phospholipid, a PEGylated lipid and a sterol. Each component has specialized functions in LNP stability and activity. The sterol and the PEGylated lipid are vital for LNP structure and stability, whereas the phospholipid can contribute to stability and endosomal escape. The cationic or ionizable lipid in turn is considered the main driver of activity and tolerability by governing mRNA encapsulation, cellular uptake and endosomal escape. Although effective nucleic acid delivery vehicles, LNPs can induce dose limiting toxicities, such as Complement Activation Related Pseudo-allergy, inflammatory cytokine release and cellular toxicities by accumulation of non-degradable ionizable lipids into cellular membranes. Further improvements in cationic or ionizable lipid chemistries are hence needed to improve efficacy and safety of LNP delivered nucleic acid drugs.

Accordingly, the present invention relates to a new class of ionizable lipids as defined by the present set of claims, which have improved characteristics over the currently available classes of ionizable lipids.

### SUMMARY OF THE INVENTION

In a first aspect, the present invention provides a lipid, in particular an ionizable lipid represented by formula (I)

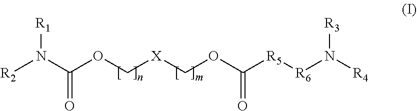

wherein
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

In a specific embodiment, the present invention provides a lipid, in particular an ionizable lipid as defined herein and being represented by anyone of formula (Ia), (Ib) and (Ic)

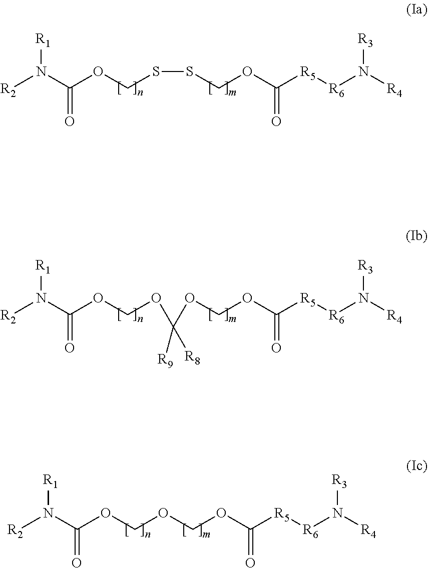

wherein
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

In yet a further embodiment, the present invention provides a lipid, in particular an ionizable lipid as defined herein and being represented by any one of formula (II) or (III)

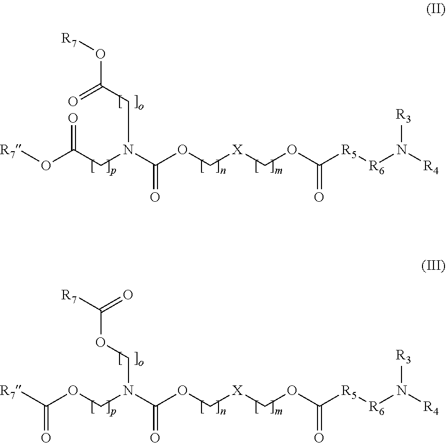

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
o and p are each independently an integer selected from 1 to 10;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—;
R₆ is —C₁₋₆alkylene-; and
wherein o and p differ from each other; or wherein R₇ and R₇″ differ from each other.

The present invention further provides a lipid, in particular an ionizable lipid as defined herein and being represented by formula (IIa)

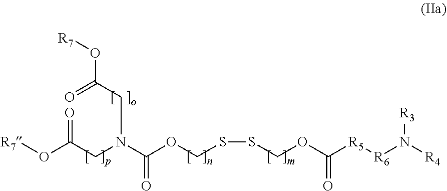

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH; each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
R₅ is selected from —NH— and —O—;
R₆ is —C₁₋₆alkylene-; and
wherein o and p differ from each other; and/or wherein R₇ and R₇″ differ from each other.

The present invention further provides a lipid, in particular an ionizable lipid as defined herein and being selected from the list comprising:

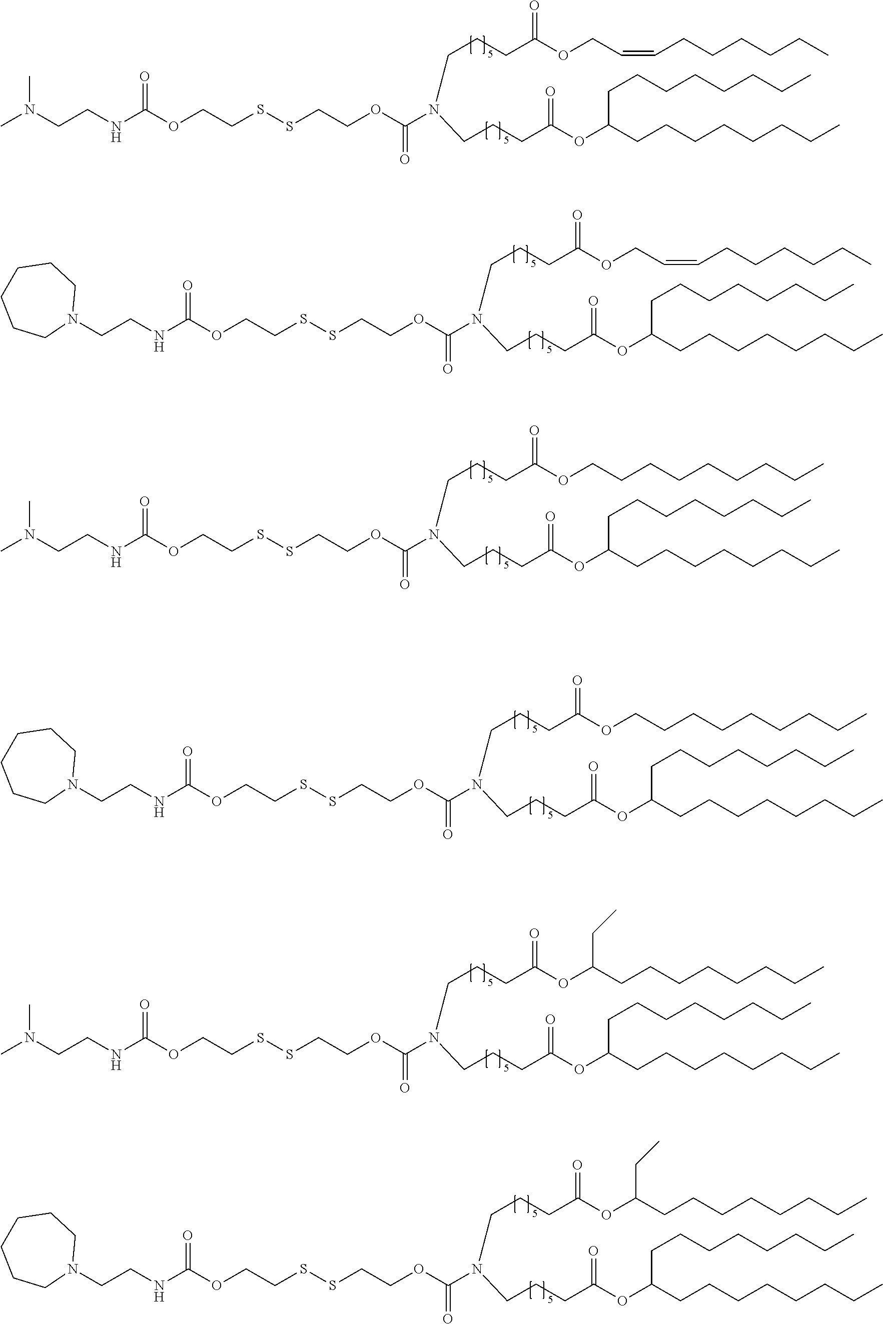

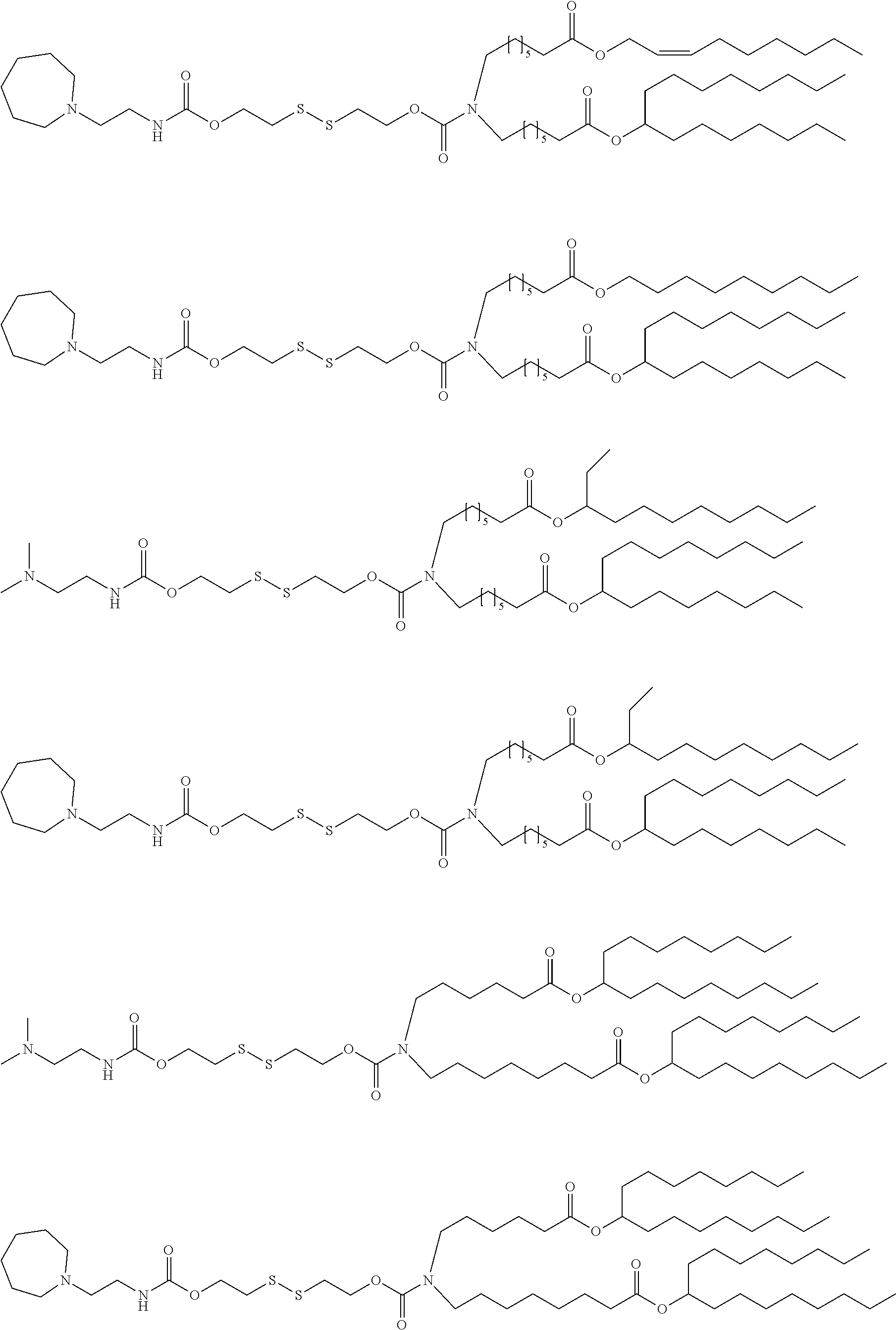

In yet a further embodiment, the present invention provides a lipid, in particular an ionizable lipid as defined herein; wherein the total number of C atoms in R₁ and R₂ together is at least 14.

The present invention further provides a lipid, in particular an ionizable lipid as defined herein; wherein the total number of C atoms in R₇ and R₇″ together is at least 12.

In a further embodiment, the present invention provides a lipid, in particular an ionizable lipid as defined herein; wherein m and n are the same, being an integer selected from 1, 2, 3 and 4; preferably 2.

The present invention further provides a lipid, in particular an ionizable lipid as defined herein; wherein RN is —NH—.

In a further aspect, the present invention provides a lipid nanoparticle or lipid nanoparticle composition comprising a lipid, in particular an ionizable lipid as defined herein. Said nanoparticle composition may further comprise a phospholipid, a sterol and a PEG lipid.

In yet a further embodiment of the present invention, the lipid nanoparticle or lipid nanoparticle composition as defined herein further comprises an active agent, in particular a nucleic acid, preferably mRNA.

In a further aspect, the present invention provides the use of a lipid, in particular an ionizable lipid as defined herein in the manufacture of a lipid nanoparticle or lipid nanoparticle composition.

In a final aspect, the present invention provides a pharmaceutical composition comprising a lipid nanoparticle or lipid nanoparticle composition as defined herein and a pharmaceutically acceptable agent.

The invention also provides the pharmaceutical compositions as defined herein for use in human and/or veterinary medicine.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention.

In this regard no attempt is made to show structural details of the invention in more detail than is necessary for a fundamental understanding of the invention. The description taken with the drawings making apparent to those skilled in the art how the several forms of the invention may be embodied in practice.

FIG. 1: In vivo bioluminescence measurement at 4 and 24 hours post injection with the respective mRNA LNPs (5 μg mRNA, 100 μL volume; TBS buffer) or control buffer.

FIG. 2: IgG1 antibody titers against HA at d21 (prime) and d35 (boost) post injection with the respective mRNA LNPs (2 μg mRNA, TBS buffer) or control buffer.

FIG. 3: hEPO expression at different timepoints post injection with the respective mRNA LNPs (10 μg mRNA, TBS buffer) or control buffer. A) Area under curve analysis. B) hEPO serum concentrations.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless a context dictates otherwise, asterisks are used herein to indicate the point at which a mono- or bivalent radical depicted is connected to the structure to which it relates and of which the radical forms part.

As already mentioned hereinbefore, in a first aspect the present invention provides a lipid, in particular an ionizable lipid represented by formula (I)

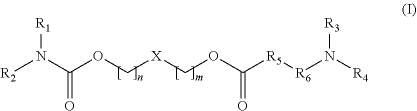

wherein
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

Accordingly, the present invention also provides a lipid, in particular an ionizable lipid represented by anyone of formula (Ia), (Ib) and (Ic)

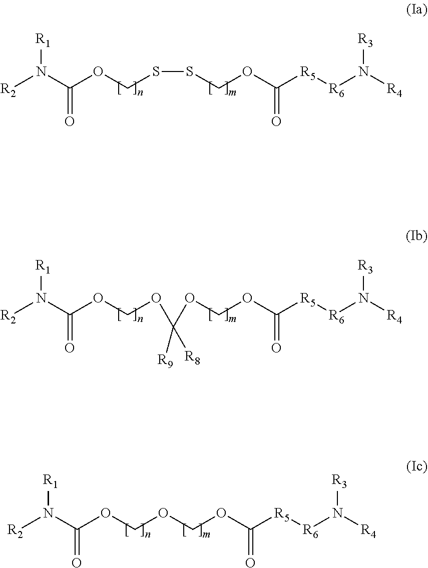

wherein
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

When describing the compounds/lipids of the present invention, the terms used are to be construed in accordance with the following definitions, unless a context dictates otherwise:

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 20 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, nonadecyl and its isomers, eicosanyl and its isomers.

The term “optionally substituted alkyl” refers to an alkyl group optionally substituted with one or more substituents (for example 1 to 4 substituents, for example 1, 2, 3, or 4 substituents) at any available point of attachment. Non-limiting examples of such substituents include esters, carboxylic acids, alkyl moieties, alkene moieties, alkyne moieties, . . . and the like.

In the context of the present invention, the alkyl, alkene and alkyne moieties as defined herein may also further comprise one or more heteroatoms, in that for example a C atom in an alkyl, alkene or alkyne chain is replaced by a heteroatom, such as selected from N, S or O.

The term “alkenyl” or “alkene”, as used herein, unless otherwise indicated, means straight-chain, cyclic, or branched-chain hydrocarbon radicals containing at least one carbon-carbon double bond. Examples of alkenyl radicals include ethenyl, propenyl, isopropenyl, butenyl, isobutenyl, pentenyl, hexenyl, hexadienyl, be it in the terminal or internal positions and the like. Generally alkenyl or alkene moieties of the present invention comprise from 2 to 20 C atoms. An optionally substituted alkenyl refers to an alkenyl having optionally one or more substituents (for example 1, 2, 3 or 4), selected from those defined above for substituted alkyl. The term “alkynyl” or “alkyne”, as used herein, unless otherwise indicated, means straight-chain or branched-chain hydrocarbon radicals containing at least one carbon-carbon triple bond. Examples of alkynyl radicals include ethynyl, E- and Z-propynyl, isopropynyl, E- and Z-butynyl, E- and Z-isobutynyl, E- and Z-pentynyl, E, Z-hexynyl, and the like. Generally alkenyl or alkene moieties of the present invention comprise from 2 to 20 C atoms. An optionally substituted alkynyl refers to an alkynyl having optionally one or more substituents (for example 1, 2, 3 or 4), selected from those defined above for substituted alkyl.

The term “cycloalkyl” by itself or as part of another substituent is a cyclic alkyl group, that is to say, a monovalent, saturated, or unsaturated hydrocarbyl group having 1, 2, or 3 cyclic structure. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 15 atoms. Examples of cycloalkyl groups include but are not limited to cyclopropyl, cyclobutyl, cyclopentyl, cyclohexyl, cycloheptyl, cyclooctyl, cyclononyl, adamantanyl and cyclodecyl with cyclopropyl being particularly preferred. An “optionally substituted cycloalkyl” refers to a cycloalkyl having optionally one or more substituents (for example 1 to 3 substituents, for example 1, 2, 3 or 4 substituents), selected from those defined above for substituted alkyl.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene. Similarly, where alkenyl groups as defined above and alkynyl groups as defined above, respectively, are divalent radicals having single bonds for attachment to two other groups, they are termed “alkenylene” and “alkynylene” respectively.

The term “heterocycle” as used herein by itself or as part of another group refers to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituents (for example 1 to 4 substituents, or for example 1, 2, 3 or 4), selected from those defined above for substituted alkyl. Non-limiting examples of heterocycle comprise: piperidinyl, pyrrolidinyl, azepanyl, morpholinyl, . . . .

The term “aryl” (herein also referred to as aromatic heterocycle) as used herein refers to a polyunsaturated, aromatic hydrocarbyl group having a single ring (i.e. phenyl) or multiple aromatic rings fused together (e.g. naphthalene or anthracene) or linked covalently, typically containing 6 to 10 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, . . . .

The aryl ring or heterocycle as defined herein can optionally be substituted by one or more substituents (for example 1 to 5 substituents, for example 1, 2, 3 or 4) at any available point of attachment. Non-limiting examples of such substituents are selected from halogen, hydroxyl, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkoxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R^{a}, alkylthio, carboxyl, and the like, wherein R^{a} is alkyl or cycloalkyl.

Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: piridinyl, azepinyl, . . . .

An “optionally substituted heteroaryl” refers to a heteroaryl having optionally one or more substituents (for example 1 to 4 substituents, for example 1, 2, 3 or 4), selected from those defined above for substituted aryl.

The term “oxo” as used herein refers to the group ═O.

The term “alkoxy” or “alkyloxy” as used herein refers to a radical having the Formula —OR^{b} wherein R^{b} is alkyl. Preferably, alkoxy is C₁-C₁₀ alkoxy, C₁-C₆ alkoxy, or C₁-C₄ alkoxy. Non-limiting examples of suitable alkoxy include methoxy, ethoxy, propoxy, isopropoxy, butoxy, isobutoxy, sec-butoxy, tert-butoxy, pentyloxy and hexyloxy. Where the oxygen atom in an alkoxy group is substituted with sulfur, the resultant radical is referred to as thioalkoxy. “Haloalkoxy” is an alkoxy group wherein one or more hydrogen atoms in the alkyl group are substituted with halogen. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy; trichloromethoxy, 2-bromoethoxy, pentafluoroethyl, 3,3,3-trichloropropoxy, 4,4,4-trichlorobutoxy.

The term “carboxy” or “carboxyl” or “hydroxycarbonyl” by itself or as part of another substituent refers to the group —CO₂H. Thus, a carboxyalkyl is an alkyl group as defined above having at least one substituent that is —CO₂H.

The term “alkoxycarbonyl” by itself or as part of another substituent refers to a carboxy group linked to an alkyl radical i.e. to form —C(═O)OR^{e}, wherein R^{e} is as defined above for alkyl.

The term “alkylcarbonyloxy” by itself or as part of another substituent refers to a —O—C(═O)R^{e} wherein R^{e} is as defined above for alkyl.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once, twice or thrice. Substituents may be selected from, for example, the group comprising halogen, hydroxyl, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

Furthermore, where groups are divalent, i.e. have two single bonds for attachment to two other groups, each occurrence thereof may be present in either of both directions in the molecule, even if not specifically indicated in the structural formulae or definition of R groups. For example —(C═N—NH₂)— as part of X means that X may for example be represented by —(C═N—NH₂)— or alternatively by the reverse orientation being —(NH₂—C═N)—.

In the context of the present invention, the term lipid is meant to be a chemically defined substance that is insoluble in water but soluble in amongst others alcohol, ether and chloroform. Ionizable or cationic lipids are lipids that are typically composed of three section: an amine head group, a linker moiety and a hydrophobic tail. The term “ionizable” (or alternatively cationic) in the context of a compound or lipid means the presence of any uncharged group in said compound or lipid which is capable of dissociating by yielding an ion (usually an H⁺ ion) and thus itself becoming positively charged. Alternatively, any uncharged group in said compound or lipid may yield an electron and thus becoming negatively charged.

In the context of the present invention, the linker moiety may be selected from a variety of different linkers, however, disulfide, ketal and ether linkers are particularly preferred.

Accordingly, and in order to obtain their lipid character, the compounds of the present invention comprise a lipid tail being represented by R₁ and R₂, wherein the total number of C atoms for both groups combined is, at least 8, such as at least 9, at least 10, at least 11, at least 12, at least 13, at least 14, at least 15, at least 16, at least 17, at least 18, at least 19 or at least 20.

Accordingly, in the context of the present invention, R₁ may for example contain 3 C atoms, while R₂ may contain 5 C atoms, thereby the total number of C atoms for both groups combined is at least 8. At any instance in the context of the present invention, R₁ and R₂ are not identical to each other.

Accordingly, in a specific embodiment, the present invention provides a lipid, in particular an ionizable lipid as defined herein and being in particular an ionizable lipid as defined herein and being represented by any one of formula (II) or (III)

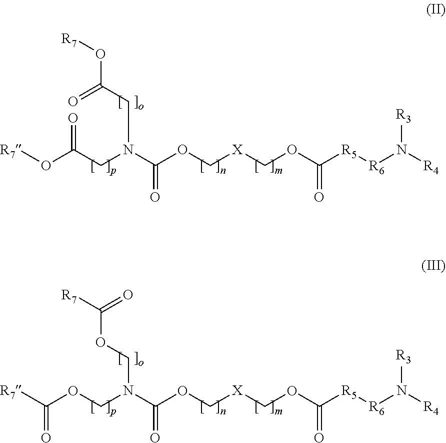

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—;
R₆ is —C₁₋₆alkylene-; and
wherein o and p differ from each other; or wherein R₇ and R₇″ differ from each other.

In yet a further embodiment, the present invention provides a lipid, in particular an ionizable lipid as defined herein and being represented by any one of formula (IIa), (IIb) or (IIc);

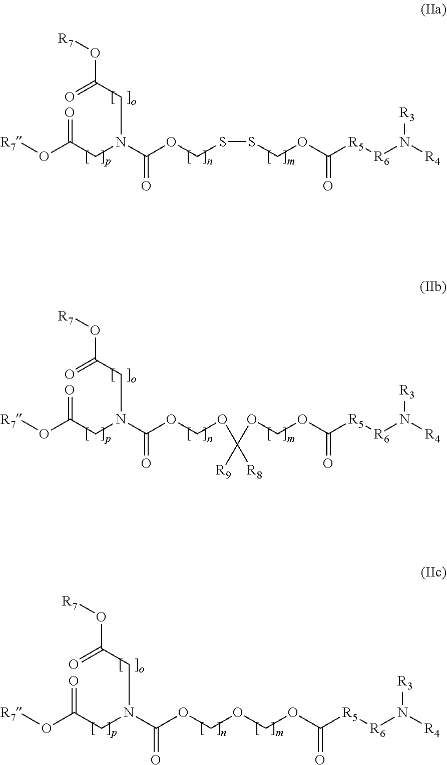

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
R₅ is selected from —NH— and —O—;
R₆ is —C₁₋₆alkylene-;
each R₈ and R₉ is independently selected from —H, —C₁₋₆alkyl and —C₃₋₆cycloalkyl;
and wherein o and p differ from each other; and/or wherein R₇ and R₇″ differ from each other.

In yet a further embodiment, the present invention provides a lipid, in particular an ionizable lipid as defined herein and being represented by any one of formula (IIIa), (IIIb) or (IIIc);

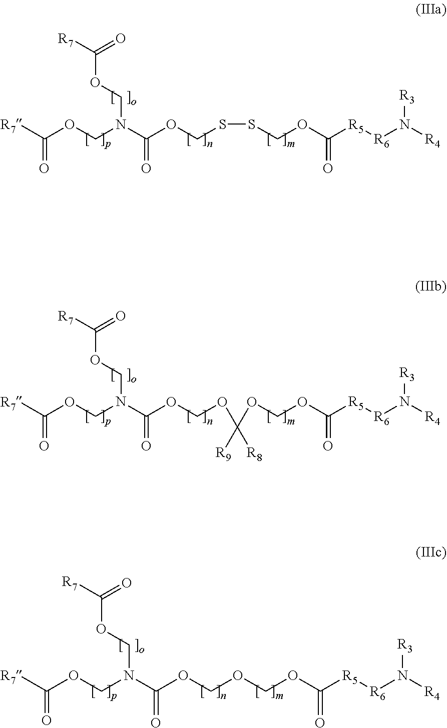

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₁₀alkenyl, —C₂₋₁₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
R₅ is selected from —NH— and —O—;
R₆ is —C₁₋₆alkylene-;
each R₈ and R₉ is independently selected from —H, —C₁₋₆alkyl and —C₃₋₆cycloalkyl;
and wherein o and p differ from each other; and/or wherein R₇ and R₇″ differ from each other.

In a further specific embodiment, the present invention provides a lipid, in particular an ionizable lipid as defined herein and being in particular an ionizable lipid as defined herein and being represented by any one of formula (IV) or (V)

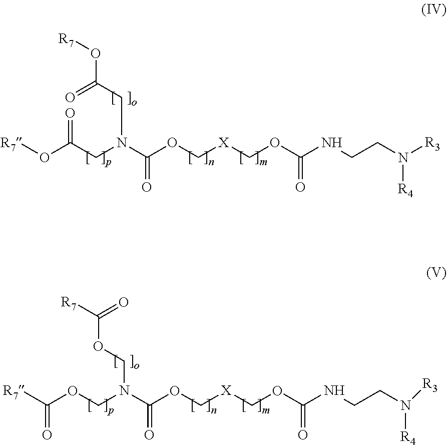

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH; each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
and wherein o and p differ from each other; or wherein R₇ and R₇″ differ from each other.

In yet a further embodiment, the present invention provides a lipid, in particular an ionizable lipid as defined herein and being represented by any one of formula (IVa), (IVb) or (IVc);

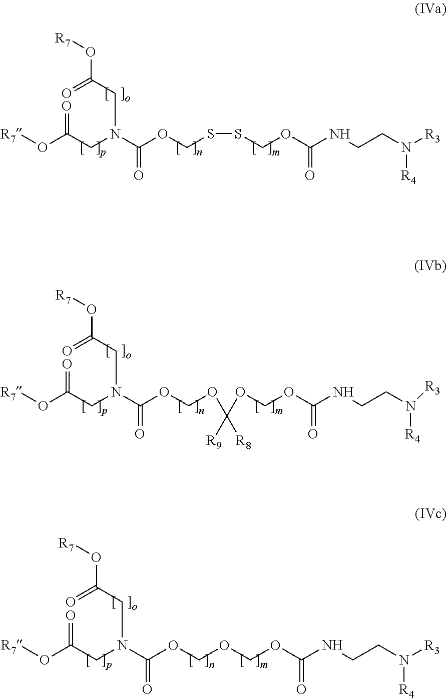

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
each R₈ and R₉ is independently selected from —H, —C₁₋₆alkyl and —C₃₋₆cycloalkyl;
and wherein o and p differ from each other; and/or wherein R₇ and R₇″ differ from each other.

In yet a further embodiment, the present invention provides a lipid, in particular an ionizable lipid as defined herein and being represented by any one of formula (Va), (Vb) or (Vc);

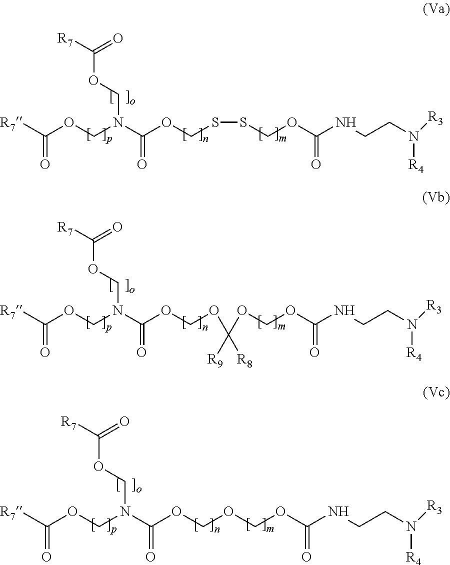

wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
and p are each independently an integer selected from 1 to 10;
each R₈ and R₉ is independently selected from —H, —C₁₋₆alkyl and —C₃₋₆cycloalkyl;
and wherein o and p differ from each other; and/or wherein R₇ and R₇″ differ from each other.

In another specific embodiment, the moiety —NR₃R₄ may be selected from the list comprising: —
—N(C₁₋₆ alkyl)₂ or an N-containing non-aromatic heterocycle using said N atom as a point of attachment. In a very specific embodiment, the moiety —NR₃R₄ may be selected from the list comprising: —N—(CH₃)₂, -pyrrolidinyl, -piperidinyl, or -azepanyl; in particular —N—(CH₃)₂, or -azepanyl.

In a very specific embodiment, the present invention provides a compound according to any of the formula (I), (II), (III), (IV), (V) and any variants (a), (b), or (c) thereof, wherein one or more of the following applies:
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

In another very specific embodiment, the present invention provides a compound according to any of the formula (I), (II), (III), (IV), (V) and any variants (a), (b), or (c) thereof, wherein one or more of the following applies:
R₁ and R₂ are each independently selected from —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered non-aromatic heterocycle;
each occurrence of R₇ is independently selected from —C₁₋₂₀alkyl, —C₂₋₂₀alkenyl, —C₂₋₂₀alkynyl;
m and n are each independently an integer selected from 1, 2, 3 and 4;
X is selected from —O—, —S—S—, —O—CR₈R₉—O—;
each R₈ and R₉ is independently selected from —H, —C₁₋₆alkyl;
R₅ is —NH—; and
R₆ is —C₁₋₆alkylene-.

The present invention further provides a lipid, in particular an ionizable lipid as defined herein and being selected from the list comprising:

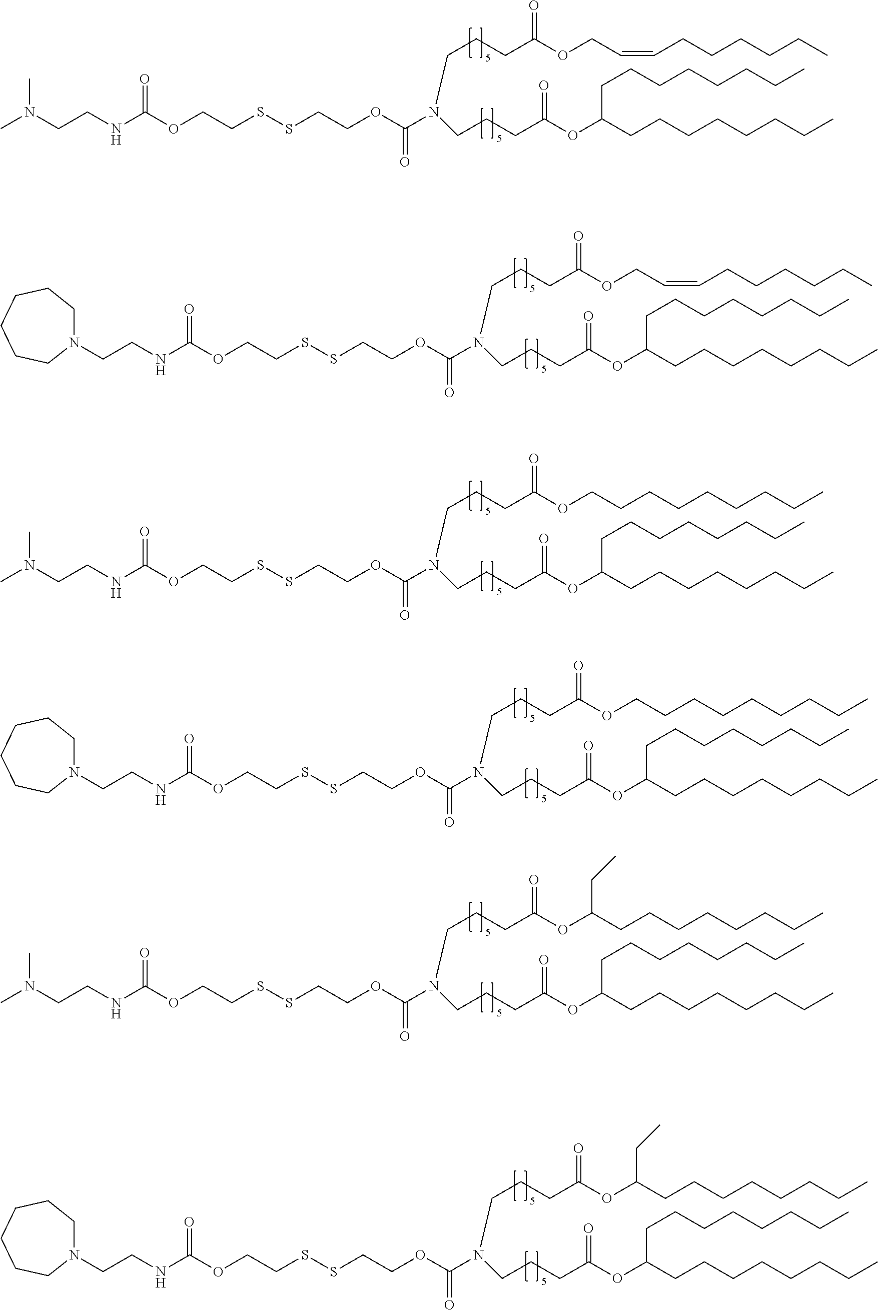

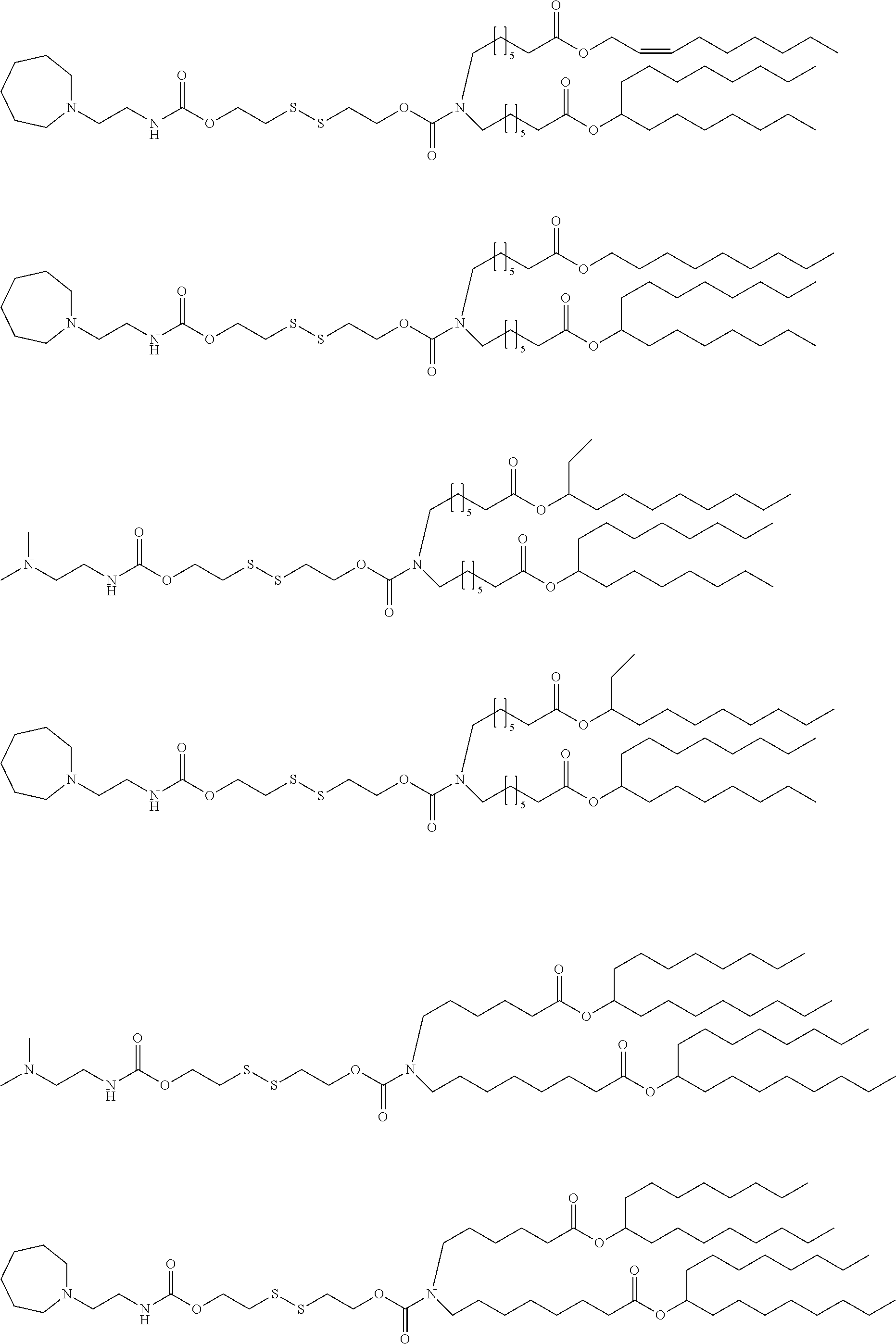

All of the lipids as defined herein may occur as different isomers/stereomers. In particular, the lipids as defined herein may occur in the trans or cis configuration, such as when they contain double bonds. In a preferred embodiment, the lipids as defined herein occur in the cis configuration. In the context of the present invention, the term ‘cis’ indicates that the functional groups are on the same side of a plane, whereas ‘trans’ means that they are on opposite sides.

In yet a further embodiment, the present invention provides a lipid, in particular an ionizable lipid as defined herein; wherein the total number of C atoms in R₁ and R₂ together is at least 14, such as at least 15, at least 17, at least 18, at least 19 or at least 20.

The present invention further provides a lipid, in particular an ionizable lipid as defined herein; wherein the total number of C atoms in R₇ and R₇″ together is at least 12.

In a further embodiment, the present invention provides a lipid, in particular an ionizable lipid as defined herein; wherein m and n are the same, being an integer selected from 1, 2, 3 and 4; such as 1 or 2 or 3 or 4; preferably 2.

The present invention further provides a lipid, in particular an ionizable lipid as defined herein; wherein R₅ is —NH—.

In a further aspect, the present invention provides a lipid nanoparticle or lipid nanoparticle composition comprising a lipid, in particular an ionizable lipid as defined herein.

In the context of the present invention, the term lipid nanoparticle (LNP), also termed solid lipid nanoparticles, is meant to be a nanoparticle comprising lipids. They are often used as a pharmaceutical drug delivery system or pharmaceutical formulation. LNPs as drug delivery vehicle were first approved in 2018, and are currently used in several candidate RNA based vaccines. A lipid nanoparticle is typically spherical with an average diameter between 10 and 1000 nanometers, and possesses a lipid core matrix that can solubilize lipophilic molecules. The term lipid is used here in a broader sense and includes triglycerides, diglycerides, monoglycerides, fatty acids, steroids (e.g. cholesterol) and waxes. Biological membrane lipids such as phospholipids, sphingomyelins, bile acids and sterols are typically used as stabilizers in LNPs.

As used herein, the term “nanoparticle” refers to any particle having a diameter making the particle suitable for systemic, in particular intravenous administration, of, in particular, nucleic acids, typically having a diameter of less than 1000 nanometers (nm), preferably less than 500 nm, even more preferably less than 200 nm, such as for example between 50 and 200 nm; preferably between 80 and 160 nm.

Accordingly, in the context of the present invention, the nanoparticles as disclosed herein further comprise one or more additional lipids either or not acting as stabilizers, such as a phospholipid, a sterol and/or a PEG lipid.

In the context of the present invention, the term “PEG lipid” or alternatively “PEGylated lipid” is meant to be any suitable lipid modified with a PEG (polyethylene glycol) group. Particularly suitable PEG lipids in the context of the present invention are characterized in being C18-PEG lipids, C14-PEG lipids (e.g. DMG-PEG or DMG-PEG2000) or C16-PEG lipids.

C18-PEG lipids contain a polyethylene glycol moiety, which defines the molecular weight of the lipids, as well as a fatty acid tail comprising 18 C-atoms. In a particular embodiment, said C18-PEG2000 lipid is selected from the list comprising: a (distearoyl-based)-PEG2000 lipid such as DSG-PEG2000 lipid (2-distearoyl-rac-glycero-3-methoxypolyethylene glycol-2000) or DSPE-PEG2000 lipid (1,2-distearoyl-sn-glycero-3-phosphoethanolamine-N-[methoxy(polyethylene glycol)-2000]); or a (dioleolyl-based)-PEG2000 lipid such as DOG-PEG2000 lipid (1,2-Dioleolyl-rac-glycerol) or DOPE-PEG2000 lipid (1,2-dioleoyl-sn-glycero-3-phosphoethanolamine-N-[amino(polyethylene glycol)-2000])

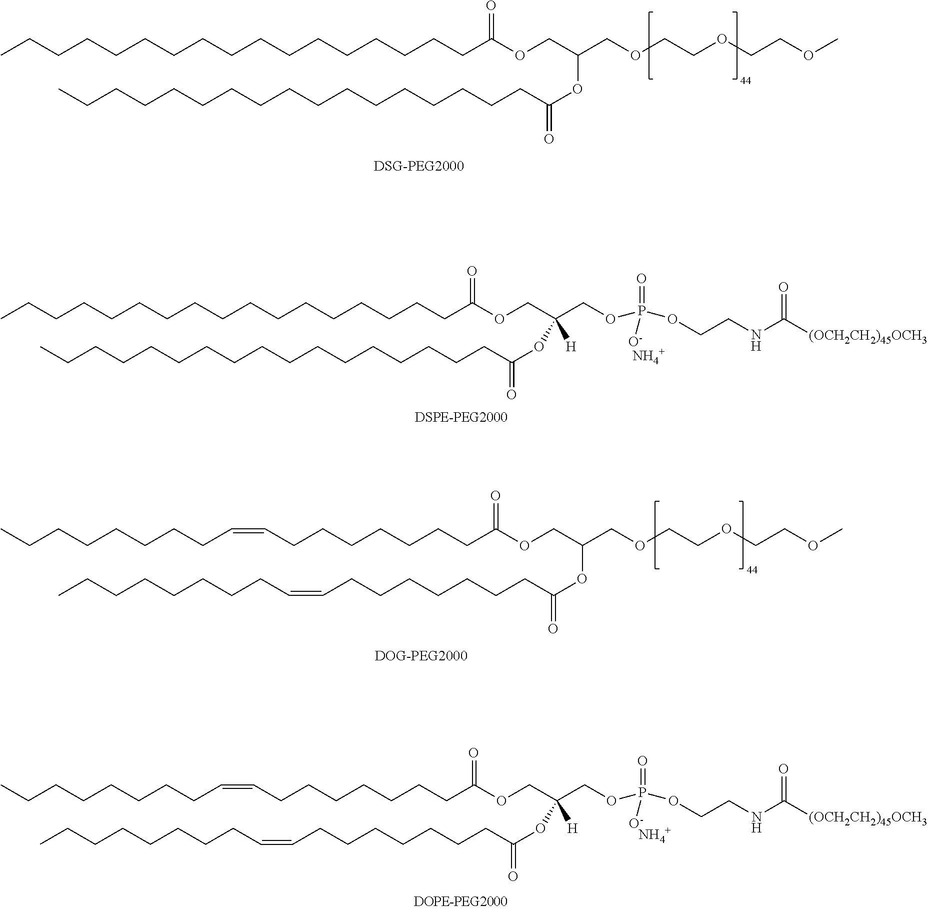

C14-PEG lipids contain a polyethylene glycol moiety, which defines the molecular weight of the lipids, as well as a fatty acid tail comprising 14 C-atoms. In a particular embodiment, said C14-PEG2000 lipid is based on dimyristoyl, i.e. having 2 C14 tails, such as selected from the list comprising: a (dimyristoyl-based)-PEG2000 lipid such as DMG-PEG2000 lipid (1,2-dimyristoyl-rac-glycero-3-methoxypolyethylene glycol-2000) or 2-Dimyristoyl-sn-Glycero-3-Phosphoethanolamine glycol-2000 (DMPE-PEG2000).

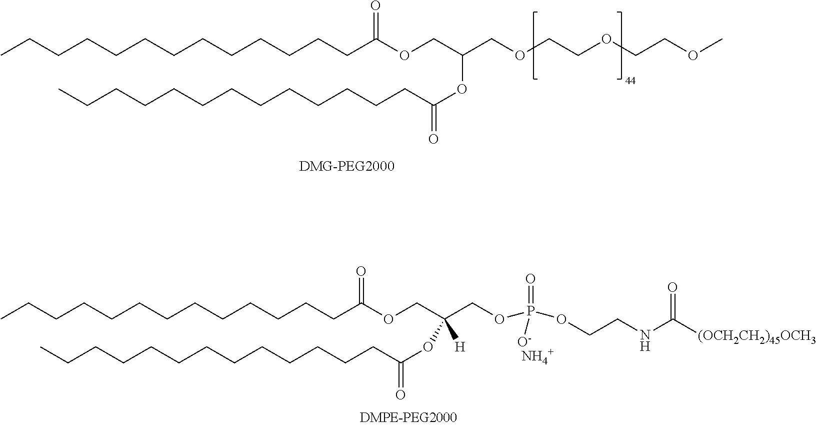

In the context of the present invention, the term “phospholipid” is meant to be a lipid molecule consisting of two hydrophobic fatty acid “tails” and a hydrophilic “head” consisting of a phosphate group. The two components are most often joined together by a glycerol molecule, hence, the phospholipid of the present invention is preferably a glycerol-phospholipid.

Furthermore, the phosphate group is often modified with simple organic molecules such as choline (i.e. rendering a phosphocholine) or ethanolamine (i.e. rendering a phosphoethanolamine).

Suitable phospholipids within the context of the invention can be selected from the list comprising: 1,2-Dioleoyl-sn-glycero-3-phosphoethanolamine (DOPE), 1,2-Dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In a more specific embodiment, said phospholipid is selected from the list comprising: 1,2-Dioleoyl-sn-glycero-3-phosphoethanolamine (DOPE), 1,2-Dioleoyl-sn-glycero-3-phosphocholine (DOPC), and mixtures thereof.

In the context of the present invention, the term “sterol”, also known as steroid alcohol, is a subgroup of steroids that occur naturally in plants, animal and fungi, or can be produced by some bacteria. In the context of the present invention, any suitable sterol may be used, such as selected from the list comprising cholesterol, ergosterol, campesterol, oxysterol, antrosterol, desmosterol, nicasterol, sitosterol and stigmasterol; preferably cholesterol.

In a specific embodiment of the present invention one or more of the following applies:
said LNP comprises about and between 35 mol % and 65 mol % of said ionizable lipid;
said LNP comprises about and between 5 mol % and 25 mol % of said phospholipid;
said LNP comprises about and between 0.5 mol % and 3.0 mol % of said PEG lipid;
balanced by the amount of said sterol.

In yet a further embodiment of the present invention, the lipid nanoparticle or lipid nanoparticle composition as defined herein further comprises a cargo molecule such as a pharmaceutically active agent (e.g. small molecule) or a biomolecule, such as a peptide, protein or a nucleic acid. In a particular embodiment, the cargo may be a nucleic acid, such as DNA or RNA; preferably mRNA. In another particular embodiment, the cargo may be a TLR agonist, such as for example the TLR3 agonist polyl:C, or the TLR9 agonist CpG.

Prior to being loaded in the lipid nanoparticles, the cargo molecules may further be modified to induce an overall polyanionic nature to the molecules. This can for example be done by bonding them to a Glu10 moiety as exemplified in the examples part. The Glu10 moiety is a moiety of 10 glutamic acids which increases the polyanionic nature of the molecule to which it is attached.

Accordingly, the lipid nanoparticles and lipid nanoparticle compositions of the present invention are particularly suitable for the intracellular delivery of their cargo molecules. Hence, the present invention provides the use of the lipid nanoparticles and lipid nanoparticle compositions as defined herein for the intracellular delivery of cargo molecules.

In a particular embodiment, the lipid nanoparticle or lipid nanoparticle composition as defined herein further comprises a nucleic acid, preferably mRNA.

A “nucleic acid” in the context of the invention is a deoxyribonucleic acid (DNA) or preferably a ribonucleic acid (RNA), more preferably mRNA. Nucleic acids include according to the invention genomic DNA, cDNA, mRNA, recombinantly produced and chemically synthesized molecules. A nucleic acid may according to the invention be in the form of a molecule which is single stranded or double stranded and linear or closed covalently to form a circle. A nucleic acid can be employed for introduction into, i.e. transfection of cells, for example, in the form of RNA which can be prepared by in vitro transcription from a DNA template. The RNA can moreover be modified before application by stabilizing sequences, capping, and/or polyadenylation.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs. Nucleic acids may be comprised in a vector. The term “vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available.

In a further aspect, the present invention provides a pharmaceutical composition comprising one or more LNP's as defined herein and a pharmaceutically acceptable agent, such as a carrier, excipient . . . . Such pharmaceutical compositions are particularly suitable as a vaccine. Thus, the invention also provides a vaccine comprising one or more LNP's according to the present invention.

In the context of the present invention, the term “vaccine” as used herein is meant to be any preparation intended to provide adaptive immunity (antibodies and/or T cell responses) against a disease. To that end, a vaccine as meant herein contains at least one nucleic acid molecule, e.g. mRNA molecule encoding an antigen to which an adaptive immune response is mounted. This antigen can be present in the format of a weakened or killed form of a microbe, a protein or peptide, or an antigen encoding a nucleic acid. An antigen in the context of this invention is meant to be a protein or peptide recognized by the immune system of a host as being foreign, thereby stimulating the production of antibodies against is, with the purpose of combating such antigens. Vaccines can be prophylactic (example: to prevent or ameliorate the effects of a future infection by any natural or “wild” pathogen), or therapeutic (example, to actively treat or reduce the symptoms of an ongoing disease). The administration of vaccines is called vaccination.

The vaccine of the invention may be used for inducing an immune response, in particular an immune response against a disease-associated antigen or cells expressing a disease-associated antigen, such as an immune response against cancer. Accordingly, the vaccine may be used for prophylactic and/or therapeutic treatment of a disease involving a disease-associated antigen or cells expressing a disease-associated antigen, such as cancer. Preferably said immune response is a T cell response. In one embodiment, the disease-associated antigen is a tumor antigen. The antigen encoded by the RNA comprised in the nanoparticles described herein preferably is a disease-associated antigen or elicits an immune response against a disease-associated antigen or cells expressing a disease-associated antigen.

The present invention also provides the LNP's, pharmaceutical compositions and vaccines according to this invention for use in human or veterinary medicine. The use of the LNP's, pharmaceutical compositions and vaccines according to this invention for human or veterinary medicine is also intended. Finally, the invention provides a method for the prophylaxis and treatment of human and veterinary disorders, by administering the LNP's, pharmaceutical compositions and vaccines according to this invention to a subject in need thereof. Such pharmaceutical compositions are particularly suitable in various fields such as prophylactic vaccines, therapeutic vaccines, protein replacement therapies, gene editing, gene silencing, small molecule delivery, etc.

The present invention further provides the use of an LNP, a pharmaceutical composition or a vaccine according to the present invention for the immunogenic delivery of said one or more nucleic acid molecules. As such the LNP's, pharmaceutical compositions and vaccine of the present invention are highly useful in the treatment several human and veterinary disorders. Thus, the present invention provides the LNP's, pharmaceutical compositions and vaccines of the present invention for use in the treatment of cancer or infectious diseases.

The lipid nanoparticles of the present invention may be prepared in accordance with the protocols as specified in the Examples part. More generally, the LNP's may be prepared using a method comprising:
preparing a first alcoholic composition comprising said ionizable lipid, said phospholipid, said sterol, said PEG lipid, and a suitable alcoholic solvent;
preparing a second aqueous composition comprising said one or more nucleic acids and an aqueous solvent;
mixing said first and second composition in a microfluidic mixing device.

In further detail, the lipid components are combined in suitable concentrations in an alcoholic vehicle such as ethanol. Thereto, an aqueous composition comprising the nucleic acid is added, and subsequently loaded in a microfluidic mixing device.

The aim of microfluidic mixing is to achieve thorough and rapid mixing of multiple samples (i.e. lipid phase and nucleic acid phase) in a microscale device. Such sample mixing is typically achieved by enhancing the diffusion effect between the different species flows. Thereto several microfluidic mixing devices can be used, such as for example reviewed in Lee et al., 2011. A particularly suitable microfluidic mixing device according to the present invention is the NanoAssemblr from Precision Nanosystems.

Other technologies suitable for preparing the LNP's of the present invention include dispersing the components in a suitable dispersing medium, for example, aqueous solvent and alcoholic solvent, and applying one or more of the following methods: ethanol dilution method, a simple hydration method, sonication, heating, vortex, an ether injecting method, a French press method, a cholic acid method, a Ca²⁺ fusion method, a freeze-thaw method, a reversed-phase evaporation method, T-junction mixing, Microfluidic Hydrodynamic Focusing, Staggered Herringbone Mixing, and the like.

The ionizable lipids of the present invention can be prepared according to the reaction schemes provided in the examples hereinafter, but those skilled in the art will appreciate that these are only illustrative for the invention and that the compounds of this invention can be prepared by any of several standard synthetic processes commonly used by those skilled in the art of organic chemistry.

### EXAMPLES

### Preparation of the Lipids

### 1. General Information

Unless otherwise stated, all glassware was oven dried before use and all reactions were carried out under an argon atmosphere using standard Schlenk-techniques. Dry solvents were purchased from Acros Organics or Sigma-Aldrich and used without further purification. All reagents were purchased from commercial sources and were used without further purification unless otherwise stated. Reaction progress was monitored by thin layer chromatography (TLC) performed on aluminum plates coated with Kieselgel F254 with 0.2 mm thickness. Visualization was achieved by ultraviolet light (254 nm) or by staining with either ninhydrine, cerium molybdate or potassium permanganate. Flash column chromatography was performed using silica gel 60 (230-400 mesh, Merck and co.). Mass spectra were obtained using a Finnigan MAT 8200 (70 eV), an Agilent 5973 (70 eV), using electrospray ionization (ESI) or electron impact ionization (EI). All 1H NMR, 13C NMR NMR were recorded on a BrukerAV-400 in Chloroform-d1 or DMSO-d6. Chemical shifts are given in parts per million (ppm), referenced to tetramethylsilane using the solvent peak as internal standard (CDCl₃: ¹H=7.26 ppm, ¹³C=77.16 ppm; CD₃SOCD₃: ¹H=2.50 ppm, ¹³C=39.52 ppm). Coupling constants were quoted in Hz. ¹H NMR splitting patterns were designated as singlet (s), broad (brd), doublet (d), triplet (t), quartet (q), quintet (qu), pentet (p), sextet (se), septet (sep), octet (o) or combinations thereof. Splitting patterns that could not be interpreted were designated as multiplet (m).

### 2. Synthesis of Lipids

### 2.1 the General Route for the Synthesis of Lipids Represented by the Structure of Formula is Shown Below.

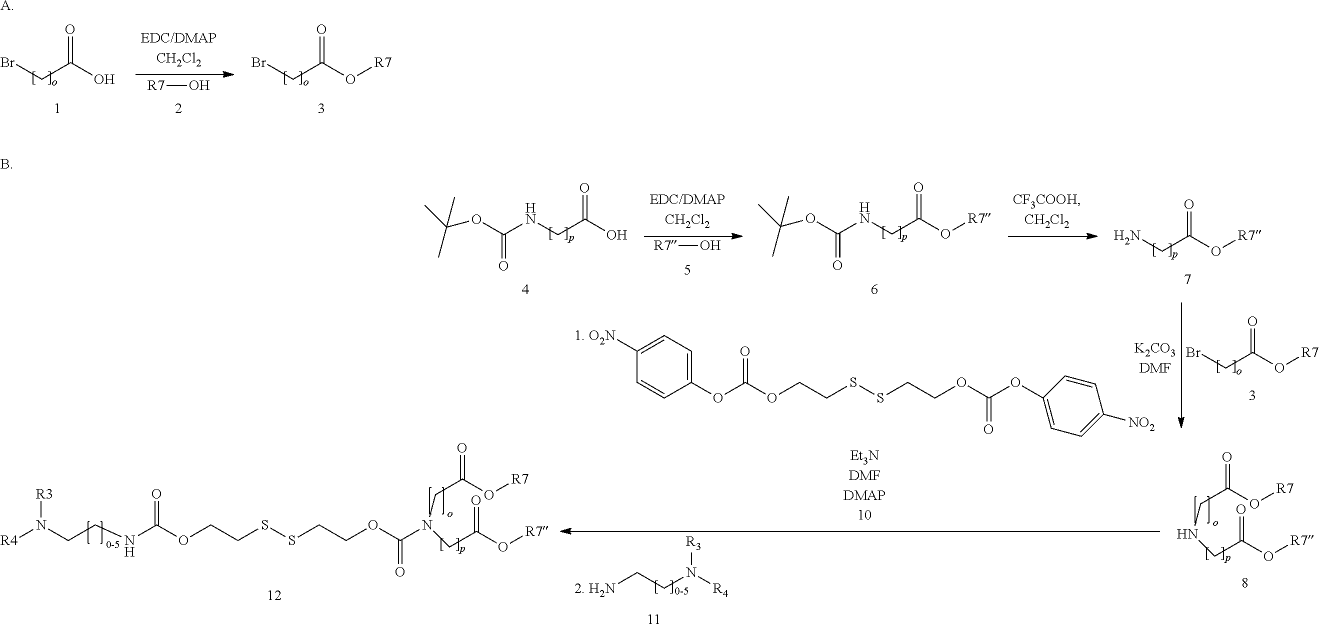

### Synthesis of Compound 3

To a stirred solution of compound 1 (1.0 eq.) and the alcohol 2 (1.2 eq) in CH₂Cl₂ at 0° C. were added N-(3-dimethylaminopropyl)-N′-ethylcarbodiimide hydrochloride (1.2 eq), N,N diisopropylethylamine (4.0 eq) and DMAP (0.2 eq.) under nitrogen (Sabnis et al, 2018). The reaction was stirred for 18 hours at room temperature. Then, the reaction was diluted with dichloromethane and washed with saturated Na₂CO₃ (aq.), brine and dried over Na₂SO₄. After filtration and concentrating in vacuo, the resulting residue was purified by silica gel column chromatography (ethyl acetate in hexane: 1 to 4%) to afford compound 3.

### Synthesis of Compound 6

To a stirred solution of compound 4 (1.0 eq.) and the alcohol 5 (1.0 eq.) in CH₂Cl₂ at 0° C. were added N-(3-dimethylaminopropyl)-N′-ethylcarbodiimide hydrochloride (1.3 eq.) Et₃N (2 eq.) and DMAP (0.2 eq.) under nitrogen (Rajappan et al, 2020). The reaction mixture was allowed to warm to room temperature and stirred for 16 hours. The organic phase was sequentially washed with sat. Na₂CO₃ (aq.), water and brine. De organic phase was dried over Na₂SO₄, filtered and concentrated in vacuo. The crude product 6 was used without further purification.

### Synthesis of Compound 7

Compound 6 was dissolved in the mixed solvent of CH₂Cl₂/CF₃COOH (10 mL/10 mL). After stirring at room temperature for 1 hour, the solvent was removed under reduced pressure and redissolved in CH₂Cl₂. The organic phase was first washed with sat. Na₂CO₃ (aq.), followed by brine and then dried over Na₂SO₄. After filtration and concentration in vacuo, the resulting residue was further purified by silica gel column chromatography (CH₂Cl₂/MeOH/NH₄OH (0.5%): 9/1) to afford compound 7.

### Synthesis of Compound 8

To a stirred solution of compound 7 in anhydrous DMF were added anhydrous K₂CO₃ (3 eq.) and a solution of compound 3 in DMF. The reaction was vigorously stirred at 80° C. for 5 h under nitrogen. After cooling to room temperature, the solids were removed by filtration and DMF was removed under reduced pressure. Finally, the crude residue was purified by silica gel column chromatography (hexane:ethyl acetate=100:0 to 85:15) to afford compound 8.

### Synthesis of Compound 12

Compound 10 (1.0 equiv.) (Amano et al., 2017), compound 8 (2.1 equiv.), DMAP (0.2 equiv.) and Et3N (5.0 equiv.) were dissolved in DMF at room temperature. The resulting mixture was stirred at room temperature for 24 h before the amine 11 (3.5 equiv.) was added. After 5 h, the solvent DMF was removed under reduced pressure, and the crude residue was redissolved in ethyl acetate. The organic phase was first washed by sat. Na2CO3 (aq.) till its color turned to off white, then washed by brine, dried over Na2SO4, filtered and concentrated. The resulting residue was purified by silica gel column chromatography (CH₂Cl2/CH₃OH=30:1 to 10:1) to afford compound 12. The names of Compound 12 are given just below their structures.

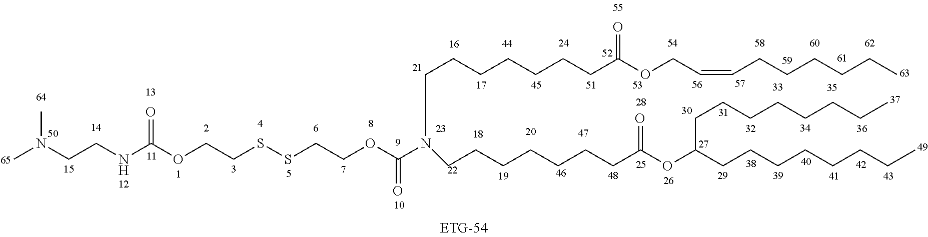 [SMILES: CCCCCC/C=C\COC(=O)CCCCCCCN(CCCCCCCC(=O)OC(CCCCCCCC)CCCCCCCC)C(=O)OCCSSCCOC(=O)NCCN(C)C]

Yield: 18% Colorless oil. ¹H-NMR (400 MHz, chloroform-d) δ 5.70-5.61 (m, 1H, H56), 5.57-5.48 (m, 2H, H57 and NH), 4.87 (qu, J=6.3 Hz, 1H, H27), 4.63 (d, J=6.9 Hz, 2H, H54), 4.38-4.28 (m, 4H, H2 & H7), 3.73 (q, J=7.0 Hz, 2H, H14), 3.26-3.12 (m, 4H, H21 & H22), 2.99-2.88 (m, 4H, H3 & H6), 2.53-2.42 (m, 2H, H15), 2.40-2.16 (m, 10H, H48, H51, H64 & H65); 2.10 (q, J=7.0 Hz, 2H, H58), 1.67-1.59 (m, 4H, H24 & H47), 1.59-1.47 (m, 8H, H16, H18, H29 & H30), 1.36-1.21 (m, 44H); 0.88 (t, J=7.00 Hz, 9H, H37, H49, H63) ppm. ¹³C-NMR (100 MHz, chloroform-d) δ 173.6 (ester C═O), 156.8 (carbamate C═O), 155.8 (carbamate C═O), 135.4 (alkene carbon), 123.4 (alkene carbon), 74.1 (C27); 62.82 (C54); 62.5, 60.2, 50.7, 45.1, 38.1, 37.9, 34.7, 34.3, 34.1, 31.8, 29.6, 29.5, 29.2, 29.1, 27.3, 26.6, 25.3, 25.1, 22.7, 14.1 (C37, C49, C63) ppm. LRMS (ESI) (m/z): calculated for [M+H]⁺ (C₅₂H₉₉N₃O₈S₂) requires 958.7, found: 958.6.

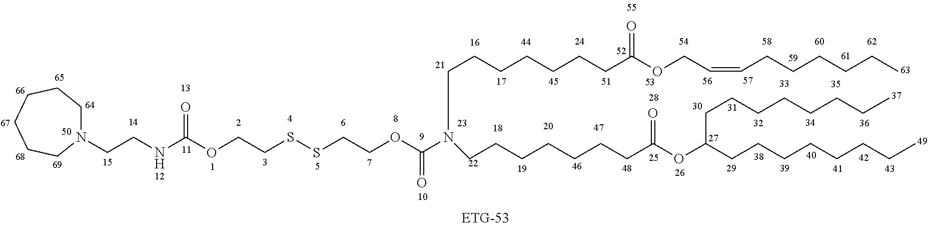 [SMILES: CCCCCC/C=C\COC(=O)CCCCCCCN(CCCCCCCC(=O)OC(CCCCCCCC)CCCCCCCC)C(=O)OCCSSCCOC(=O)NCCN1CCCCCC1]

Yield: 16% Colorless oil. ¹H-NMR (400 MHz, chloroform-d) δ 5.69-5.61 (m, 1H, H56), 5.58-5.48 (m, 1H, H57), 4.87 (qu, J=6.3 Hz, 1H, H27), 4.63 (d, J=6.9 Hz, 2H, H54), 4.37-4.29 (m, 4H, H2 & H7), 3.52-3.36 (m, 2H, H14), 3.27-3.11 (m, 4H, H64 & H69), 3.04-2.83 (m, 10H, H15, H3, H6, H21 & H22), 2.36-2.21 (m, 4H, H48, H51), 2.1 (q, J=7.1 Hz, 2H, H58), 1.88-1.76 (m, 4H, H65 & H68), 1.72-1.57 (m, 8H, H66, H67, H24 & H47), 1.56-1.45 (m, 8H, H16, H18, H29 & H30), 1.42-1.21 (m, 44H); 0.88 (t, J=6.75 Hz, 9H, H37, H49, H63) ppm. ¹³C-NMR (100 MHz, chloroform-d) δ 173.6 (ester C═O), 153.5 (carbamate C═O), 151.7 (carbamate C═O), 135.4 (alkene carbon), 123.4 (alkene carbon), 74.1 (C27); 62.7 (C54), 60.2, 57.6, 55.4, 37.8, 34.7, 34.3 34.1, 31.9, 29.5, 29.5, 29.4, 29.2, 29.1 27.5, 26.9, 26.7, 25.3, 25.1, 22.7, 14.1 (C37, C49, C63) ppm. LRMS (ESI) (m/z): calculated for [M+H]⁺ (C₅₅H₁₀₅N₃O₈S₂) requires 1012.8, found: 1012.6.

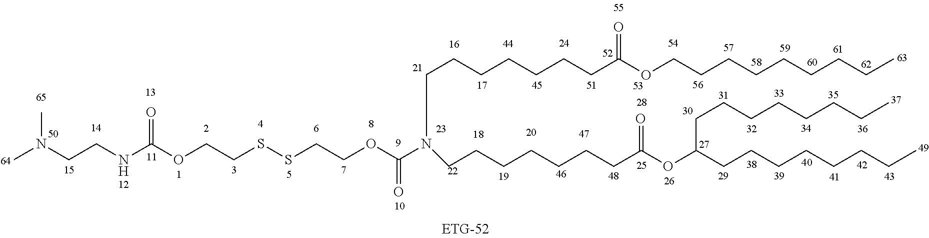 [SMILES: CCCCCCCCCOC(=O)CCCCCCCN(CCCCCCCC(=O)OC(CCCCCCCC)CCCCCCCC)C(=O)OCCSSCCOC(=O)NCCN(C)C]

Yield: 14% Colorless oil. ¹H-NMR (400 MHz, chloroform-d) δ 4.87 (qu, J=6.2 Hz, 1H, H27), 4.37-4.27 (m, 4H, H2 & H7), 4.08 (t, J=6.8 Hz, 2H, H54), 3.35-3.24 (m, 2H, H14), 3.23-3.12 (m, 4H, H21 & H22), 3.02-2.88 (m, 4H, H3 & H6), 2.50-2.40 (m, 2H, H15), 2.35-2.24 (m, 10H, H48, H51, H64 & H65), 1.71-1.58 (m, 6H, H56, H24 & H47), 1.57-1.44 (m, 8H, H16, H18, H29 & H30), 1.42-1.21 (m, 48H), 0.89 (t, J=6.75 Hz, 9H, H37, H49, H63) ppm. ¹³C-NMR (100 MHz, chloroform-d) δ 173.6 (ester C═O), 156.4 (carbamate C═O), 155.8 (carbamate C═O), 74.1 (C27), 64.4, 62.8, 62.5, 51.8, 45.11, 38.0, 37.9, 34.7, 34.3, 34.1, 31.9, 29.5, 29.2, 29.1, 28.6, 26.7, 25.9, 25.3, 25.1, 24.9, 22.7, 14.1 (C37, C49, C63) ppm. LRMS (ESI) (m/z): calculated for [M+H]⁺ (C₅₂H₁₀₁N₃O₈S₂) requires 960.7, found: 960.6

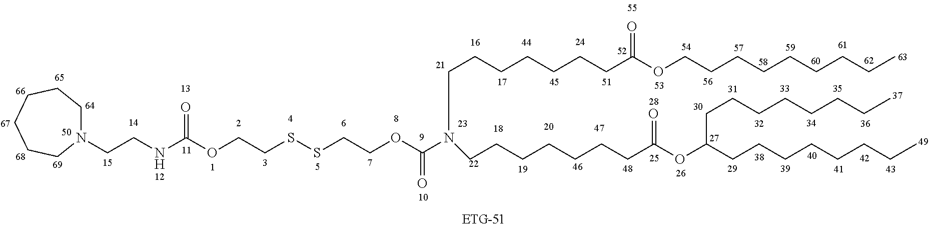 [SMILES: CCCCCCCCCOC(=O)CCCCCCCN(CCCCCCCC(=O)OC(CCCCCCCC)CCCCCCCC)C(=O)OCCSSCCOC(=O)NCCN1CCCCCC1]

Yield: 19% Colorless oil. ¹H-NMR (400 MHz, chloroform-d) δ 4.87 (qu, J=6.3 Hz, 1H, H27), 4.37-4.29 (m, 4H, H2 & H7), 4.11-4.1 (t, J=6.9 Hz, 2H, H54), 3.47-3.32 (m, 2H, H14), 3.26-3.10 (m, 4H, H64 & H69), 2.99-2.90 (m, 4H, H21 & H22), 3.04-2.83 (m, 4H, H3 & H6), 2.88-2.76 (m, 2H, H15), 2.35-2.24 (m, 4H, H48, H51), 1.85-1.71 (m, 4H, H65 & H68), 1.69-1.59 (m, 10H, H56, H66, H67, H24 & H47), 1.55-1.44 (m, 8H, H16, H18, H29 & H30), 1.42-1.21 (m, 48H), 0.89 (t, J=6.75 Hz, 9H, H37, H49, H63) ppm. ¹³C-NMR (100 MHz, chloroform-d) δ 173.6 (ester C═O), 155.7 (carbamate C═O), 149.1 (carbamate C═O), 74.1 (C27), 64.4, 64.0, 62.8, 62.7, 59.4, 55.3, 52.9, 52.8, 50.3, 50.0; 37.9, 34.7, 34.3, 34.1, 31.9, 29.5, 29.2, 29.1, 28.6, 28.1 26.9, 26.7, 25.9, 25.3, 25.1 24.9, 22.7 14.1 (C37, C49, C63) ppm. LRMS (ESI) (m/z): calculated for [M+H]⁺ (C₅₆H₁₀₇N₃O₈S₂) requires 1014.8, found: 1014.6.

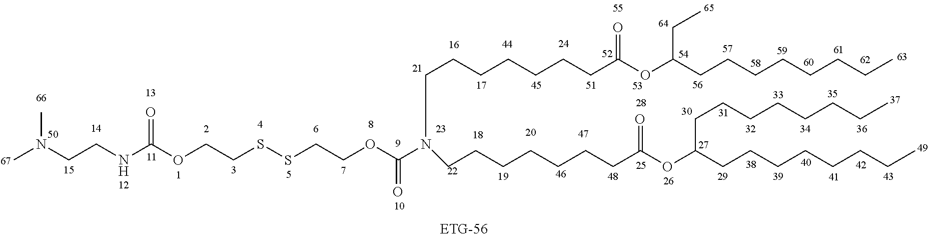 [SMILES: CCCCCCCCC(CC)OC(=O)CCCCCCCN(CCCCCCCC(=O)OC(CCCCCCCC)CCCCCCCC)C(=O)OCCSSCCOC(=O)NCCN(C)C]

Yield: 15% Colorless oil. ¹H-NMR (400 MHz, chloroform-d) δ 4.91-4.77 (m, 2H, H27 & H54), 4.36-4.28 (m, 4H, H2 & H7), 3.40-3.33 (m, 2H, H14), 3.24-3.13 (m, 4H, H21 & H22), 2.99-2.90 (m, 4H, H3 & H6), 2.68-2.57 (brd, 2H, H15), 2.44-2.36 (brd, 4H, H48, H51), 2.33-2.22 (m, 6H, H66, H67), 1.68-1.56 (m, 4H, H24 & H47), 1.57-1.44 (m, 12H, H16, H18, H29, H30, H56, H64), 1.42-1.21 (m, 48H), 0.89 (t, J=6.75 Hz, 9H, H37, H49, H63) ppm. ¹³C-NMR (100 MHz, chloroform-d) δ 173.7 (ester C═O), 162.8 (carbamate C═O), 155.8 (carbamate C═O), 75.4 (C27), 74.1, 62.8, 62.6, 58.0, 44.8, 37.9, 34.7, 34.1, 33.6, 31.8, 29.5, 29.5, 29.2; 29.1, 26.9, 26.7, 25.3, 25.1, 22.7, 14.1 (C37, C49, C63) 9.60 (C65) ppm. LRMS (ESI) (m/z): calculated for [M+H]⁺ (C₅₄H₁₀₅N₃O₈S₂) requires 988.7, found: 988.6.

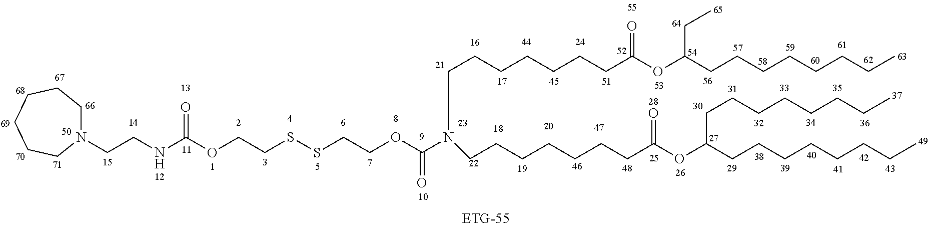 [SMILES: CCCCCCCCC(CC)OC(=O)CCCCCCCN(CCCCCCCC(=O)OC(CCCCCCCC)CCCCCCCC)C(=O)OCCSSCCOC(=O)NCCN1CCCCCC1]

Yield: 17% Colorless oil. ¹H-NMR (400 MHz, chloroform-d) δ 4.91-4.77 (m, 2H, H27 & H54), 4.36-4.28 (m, 4H, H2 & H7), 3.40-3.33 (brd, 2H, H14), 3.24-3.13 (m, 4H, H66 & H71), 2.99-2.90 (m, 4H, H21 & H22), 2.88-2.76 (brd, 6H, H15, H3 & H6), 2.36-2.22 (m, 4H, H48, H51), 1.87-1.70 (brd, 4H, H67, H70), 1.69-1.57 (m, 8H, H24, H47, H68, H69), 1.57-1.44 (m, 12H, H16, H18, H29, H30, H56, H64), 1.41-1.20 (m, 48H), 0.89 (t, J=6.75 Hz, 12H, H37, H49, H63 & H65) ppm. ¹³C-NMR (100 MHz, chloroform-d) δ 173.6 (ester C═O), 164.3 (carbamate C═O), 155.8 (carbamate C═O), 75.2 (C27), 74.1, 62.8, 62.7, 55.3, 52.9, 50.7, 37.9, 34.7, 34.1, 33.61, 31.9, 29.6, 29.5, 29.2, 29.1, 26.9, 26.7, 25.3, 25.1, 22.7, 14.1 (C37, C49, C63) 9.60 (C65) ppm. LRMS (ESI) (m/z): calculated for [M+H]⁺ (C₅₈H₁₁₁N₃O₈S₂) requires 1042.8, found: 1042.7.

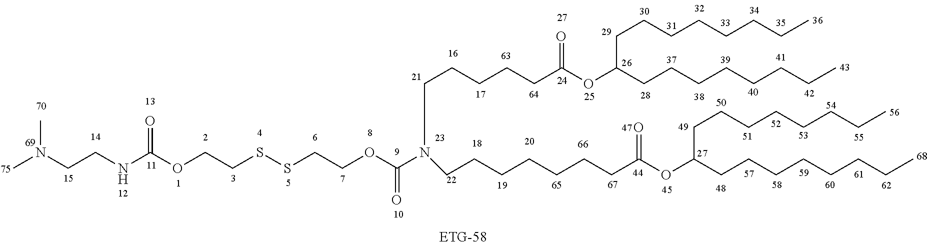 [SMILES: CCCCCCCCC(CCCCCCCC)OC(=O)CCCCCCCN(CCCCCC(=O)OC(CCCCCCCC)CCCCCCCC)C(=O)OCCSSCCOC(=O)NCCN(C)C]

Yield: 13% Colorless oil. ¹H-NMR (400 MHz, chloroform-d) δ 4.87 (qu, J=6.3, 2H, H26 & H46), 4.37-4.29 (m, 4H, H2 & H7), 3.33-3.25 (m, 2H, H14), 3.24-3.14 (m, 4H, H21 & H22), 3.00-2.88 (m, 4H, H3 & H6), 2.52-2.42 m, 2H, H15) 2.36-2.22 (m, 10H, H64, H67, H70 & H75), 1.68-1.57 (m, 4H, H63, H66), 1.57-1.47 (m, 12H, H16, H18, H28, H29, H48, H49), 1.41-1.20 (m, 56H), 0.89 (t, J=6.8 Hz, 12H, H36, H43, H56 & H68) ppm. ¹³C-NMR (100 MHz, chloroform-d) δ 173.6 (ester C═O), 163.3 (carbamate C═O), 155.8 (carbamate C═O), 74.2 (C27), 74.1, 62.9, 58.1, 53.4, 45.1, 37.9, 34.7, 34.6, 34.1, 31.9, 29.6, 29.5, 29.3, 29.2, 29.1, 26.4, 25.3, 25.1, 24.9, 22.6, 14.1 (C36, C43, C56 & C68) ppm. LRMS (ESI) (m/z): calculated for [M+H]⁺ (C₅₈H₁₁₃N₃O₈S₂) requires 1044.8, found: 1044.7.

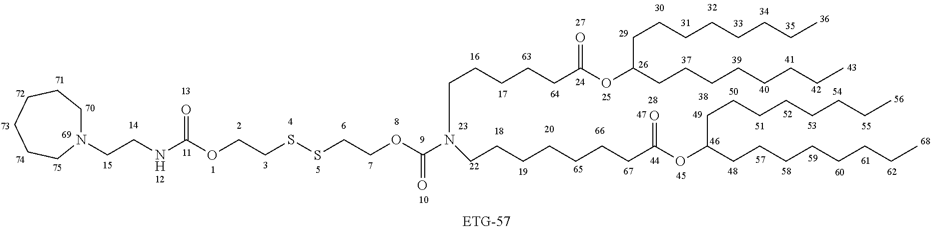 [SMILES: CCCCCCCCC(CCCCCCCC)OC(=O)CCCCCCCN(CCCCCC(=O)OC(CCCCCCCC)CCCCCCCC)C(=O)OCCSSCCOC(=O)NCCN1CCCCCC1]

Yield: 13% Colorless oil. ¹H-NMR (400 MHz, chloroform-d) δ 4.87 (qu, J=6.3, 2H, H26 & H46), 4.37-4.27 (m, 4H, H2 & H7), 3.33-3.25 (brd, 2H, H14), 3.24-3.14 (brd, 4H, H21 & H22), 3.03-2.81 (m, 10H, H3, H6, H71, H74, H15), 2.34-2.24 (m, 4H, H64, H67), 1.87-1.74 (brd, 4H, H71 & H74), 1.72-1.58 (m, 8H, H63, H66, H72, H73), 1.57-1.46 (m, 12H, H16, H18, H28, H29, H48, H49), 1.38-1.19 (m, 56H), 0.89 (t, J=6.8 Hz, 12H, H36, H43, H56 & H68) ppm. ¹³C-NMR (100 MHz, chloroform-d) δ 173.6 (ester C═O), 163.1 (carbamate C═O), 154.7 (carbamate C═O), 74.2 (C27), 74.1, 62.9, 55.3, 53.4, 45.1, 37.9, 34.7, 34.6, 34.1, 31.9, 29.6, 29.5, 29.3, 29.2, 29.1, 26.4, 25.3, 25.1, 24.9, 22.6, 14.1 (C36, C43, C56 & C68) ppm. LRMS (ESI) (m/z): calculated for [M+H]⁺ (C₆₂H₁₁₉N₃O₈S₂) requires 1098.9, found: 1.098.8.

### Example: LNP Synthesis

mRNA Synthesis:

All mRNA's were prepared in vitro by T7-mediated transcription from linearized DNA templates (peTheRNA vector), which incorporates 5′ and 3′ UTRs and a polyA tail. The final mRNA utilizes Cap1 and 100% replacement of uridine with N1-methyl-pseudo-uridine.

Ionizable lipids of the invention were prepared according to the reaction scheme defined herein above. As a comparative example, ETG-23 corresponding to S-Ac7-DOg (see WO2022136641) was used and prepared according to the reaction schemes defined in '641.

### LNP Synthesis:

Lipid based nanoparticles were produced by microfluidic mixing of an mRNA solution in sodium acetate buffer (100 mM, pH 4) and lipid solution in a 2:1 volume ratio at a speed of 16 mL/min using the NanoAssemblr Benchtop (Precision Nanosystems). LNPs were produced at a standard molar ratio ionizable lipid/DSPC (Avanti Polar Lipids)/cholesterol (Sigma)/DMG-PEG2000 (Avanti Polar Lipids) of about 50/10/38.5/1.5. eGFP mRNA was encapsulated in all LNPs as reporter mRNA, at a mRNA/ionizable lipid molar ratio of 1/10. LNPs were dialyzed against TBS (10000 times more TBS volume than LNP volume) using slide-a-lyzer dialysis cassettes (20K MWCO, 3 mL, ThermoFisher). Size, polydispersity and zeta potential were measured with a Zetasizer Nano (Malvern). mRNA encapsulation was measured by standard Ribogreen RNA assay (Invitrogen).

### Data Analysis:

All raw data were analyzed using the Graph Pad Prism version 9 software.

TABLE 1

| ionizable lipid | size | PDI | % encap |
|---|---|---|---|
| Comparative example ETG-23 | 75.97 | 0.082 | 97 |
| ETG-51 | 111.97 | 0.119 | 96 |
| ETG-52 | 90.70 | 0.099 | 97 |
| ETG-53 | 103.23 | 0.085 | 97 |
| ETG-54 | 90.30 | 0.150 | 98 |
| ETG-55 | 93.73 | 0.123 | 96 |
| ETG-56 | 86.26 | 0.120 | 98 |
| ETG-57 | 109.10 | 0.114 | 95 |
| ETG-58 | 101.50 | 0.086 | 98 |

### Example 1: In Vivo Fluc mRNA Expression Upon IV mRNA LNP Injection

LNPs were produced at a standard molar ratio ionizable lipid/DSPC/cholesterol/DMG-PEG2000 of about 50/10/38.5/1.5. Firefly Luciferase (Fluc) mRNA was encapsulated in all LNPs, at an mRNA/ionizable lipid molar ratio of 1/10.

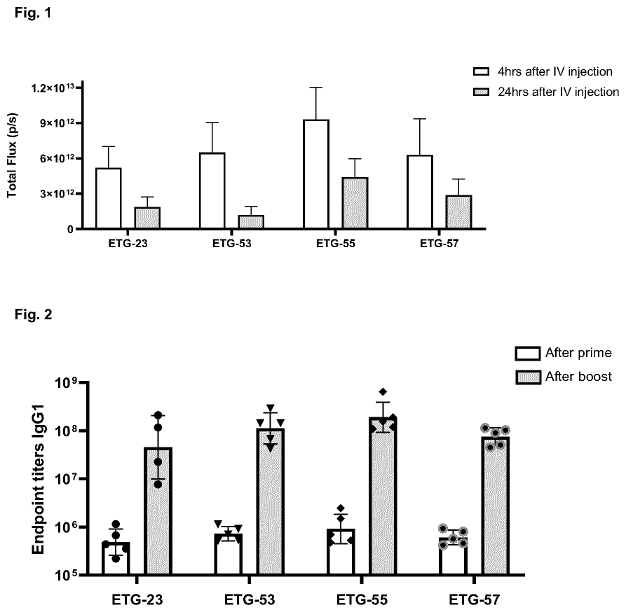

All experimental groups (BALB/c, female, 6-8 weeks old mice) received 1 intravenous injection with the respective mRNA LNPs (5 μg mRNA, 100 μL volume; TBS buffer) or control buffer. Fluc mRNA expression was assessed via in vivo bioluminescence measurement at 4 and 24 hours post injection, after intraperitoneal injection of the substrate D-luciferin. mRNA delivery by ETG-based lipids resulted in high expression levels in vivo (FIG. 1).

### Example 2: Assessment of Antibody Responses Against Influenza Hemagglutinin HA

LNPs were produced at a standard molar ratio ionizable lipid/DSPC/cholesterol/DMG-PEG2000 of about 50/10/38.5/1.5. Hemagglutinin (HA) mRNA was encapsulated in all LNPs at an mRNA/ionizable lipid molar ratio of 1/10.

At d0 and d21, BALB/c mice (female, 6 weeks old) were injected intramuscularly via bilateral injection with 50 μl of the respective mRNA LNPs (2 μg mRNA, TBS buffer) or control buffer. Blood was collected on d21 (prime) and d35 (boost), after which IgG1 antibody titers against HA were determined using an in house ELISA assay (FIG. 2).

### Example 3: In Vivo HEPO Expression after IV mRNA LNP Injection

LNPs were produced at a standard molar ratio ionizable lipid/DSPC/cholesterol/DMG-PEG2000 of about 50/10/38.5/1.5. Human erythropoietin (hEPO) mRNA was encapsulated in all LNPs at an mRNA/ionizable lipid molar ratio of 1/10.

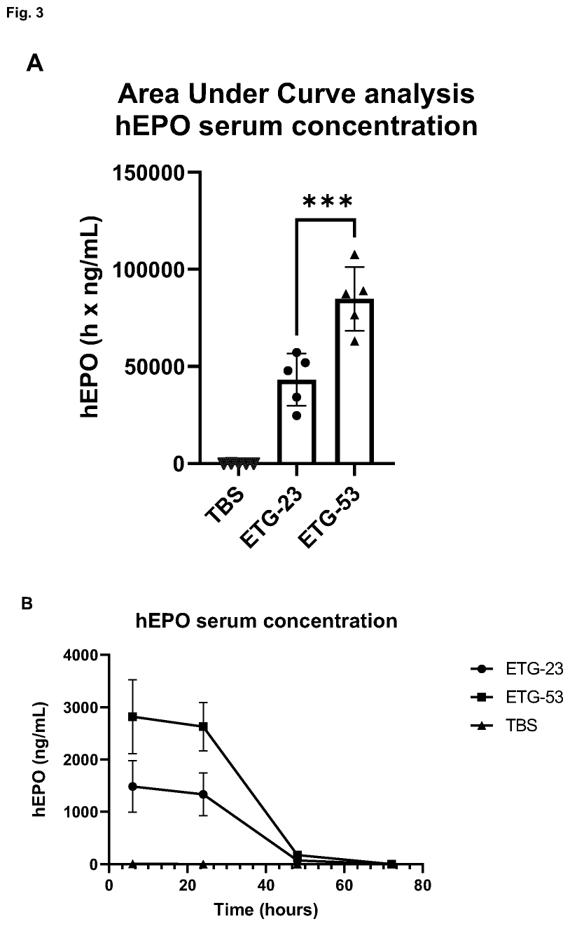

BALB/c mice (female, 6 weeks old mice) were injected intravenously in the tail vein with 200 μl of the respective mRNA LNPs (10 μg mRNA, TBS buffer) or control buffer. Blood was collected from the submandibular vein at different timepoints after which hEPO concentrations were determined using hEPO ELISA assay kit (catalog #BMS2035-2—Thermofisher Scientific), according to the manufacturer's instructions. ETG-53 shows higher hEPO expression compared to ETG-23 control (FIGS. 3 A & B).

### REFERENCES

1. Sabnis, S.; Kumarasinghe, S.; Salerno, T.; et al. A Novel Amino Lipid Series for mRNA Delivery: Improved Endosomal Escape and Sustained Pharmacology and Safety in Non-human Primates. Molecular Therapy, 2018, 26, 1509-1519
2. Rajappan, K.; Tanis, S. P.; Mukthavaram, R. et al. Property-Driven Design and Development of Lipids for Efficient Delivery of siRNA. J. Med. Chem., 2020, 63, 21, 12992-13012
3. Amano, Y.; Umezawa, N.; Sato, S.; Watanabe, H.; Umehara, T.; Higuchi, T. Activation of lysine-specific demethylase 1 inhibitor peptide by redox-controlled cleavage of a traceless linker. Bioorg. Med. Chem. 2017, 25, 1227-1234.

1. An ionizable lipid represented by formula (I) 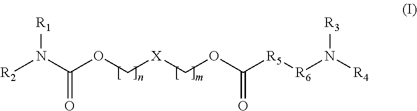
wherein
R₁ and R₂ are each independently selected from —C₁₋₂₀alkyl, —C₂₋₂₀alkenyl, and —C₂₋₂₀alkynyl;
wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

2. An ionizable lipid as defined in claim 1 and being represented by anyone of formula (Ia), (Ib) and (Ic) 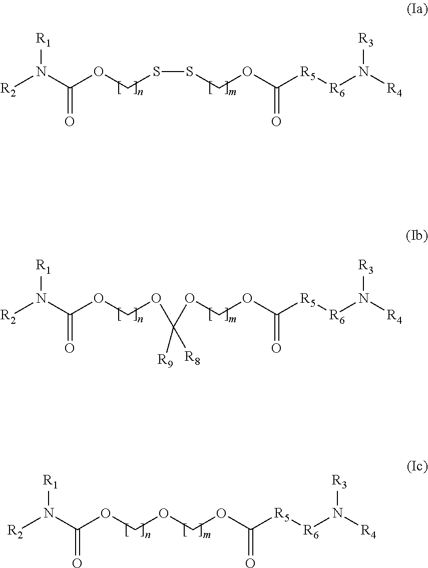
wherein
R₁ and R₂ are each independently selected from —C₁₋₂₀alkyl, —C₂₋₂₀alkenyl, and —C₂₋₂₀alkynyl; wherein each of said —C₁₋₂₀alkyl, —C₂₋₂₀alkenyl, and —C₂₋₂₀alkynyl may optionally be substituted with from 1 to 3 —O—(C═O)—R₇, —(C═O)—O—R₇, —C₁₋₂₀alkyl, —C₂₋₂₀alkenyl, and —C₂₋₂₀alkynyl;
wherein the total number of C atoms in R₁ and R₂ together is at least 8;
wherein R₁ and R₂ differ from each other;
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each R₇ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl;
m and n are each independently an integer selected from 1, 2, 3 and 4;
each R₈ and R₉ is independently selected from —H, —C₁₋₆alkyl and —C₃₋₆cycloalkyl;
R₅ is selected from —NH— and —O—; and
R₆ is —C₁₋₆alkylene-.

3. An ionizable lipid as defined in claim 1, and being represented by any one of formula (II) or (III) 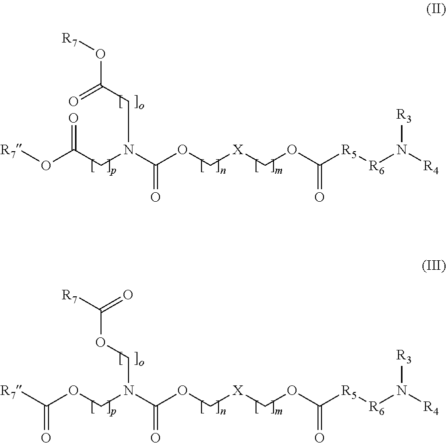
wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
o and p are each independently an integer selected from 1 to 10;
X is selected from —O—, —S—, —S—S—, —O—(C═O)—, —O—(C═O)—O—, —(C═N—NH₂)—, —O—CR₈R₉—O—, and —S—CR₈R₉—S—;
each R₈ and R₉ is independently selected from —H, —C₁₋₆alkyl and —C₃₋₆cycloalkyl;
R₅ is selected from —NH— and —O—;
R₆ is —C₁₋₆alkylene-; and
wherein o and p differ from each other; or wherein R₇ and R₇″ differ from each other.

4. An ionizable lipid as defined in claim 1 and being represented by formula (IIa) 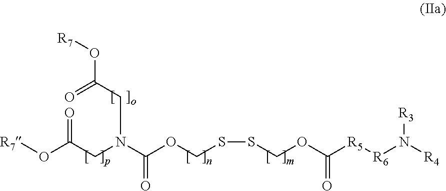
wherein
R₃ and R₄ are each independently a —C₁₋₆alkyl; or R₃ and R₄ taken together with the N atom to which they are attached form a 5-10 membered aromatic or non-aromatic heterocycle; said heterocycle may further optionally comprise one or more additional N atoms, and/or may optionally be substituted with from 1-3 substituents selected from: —C₁₋₆alkyl and —OH;
each occurrence of R₇ and R₇″ is independently selected from —C₁₋₂₀alkyl, —C₂₋₂₀alkenyl, —C₂₋₂₀alkynyl; wherein each of said —C₁₋₂₀alkyl, —C₂₋₂₀alkenyl, —C₂₋₂₀alkynyl may optionally be substituted with from 1 to 3 —O—(C═O)—R₇, —(C═O)—O—R₇, —C₁₋₂₀alkyl, —C₂₋₂₀alkenyl, —C₂₋₂₀alkynyl; and the total number of C atoms in R₇ and R₇″ together is at least 6;
m and n are each independently an integer selected from 1, 2, 3 and 4;
o and p are each independently an integer selected from 1 to 10;
R₅ is selected from —NH— and —O—;
R₆ is —C₁₋₆alkylene-; and
wherein o and p differ from each other; and/or wherein R₇ and R₇″ differ from each other.

5. An ionizable lipid as defined in any one of claims 1 to 4, and being selected from the list comprising:

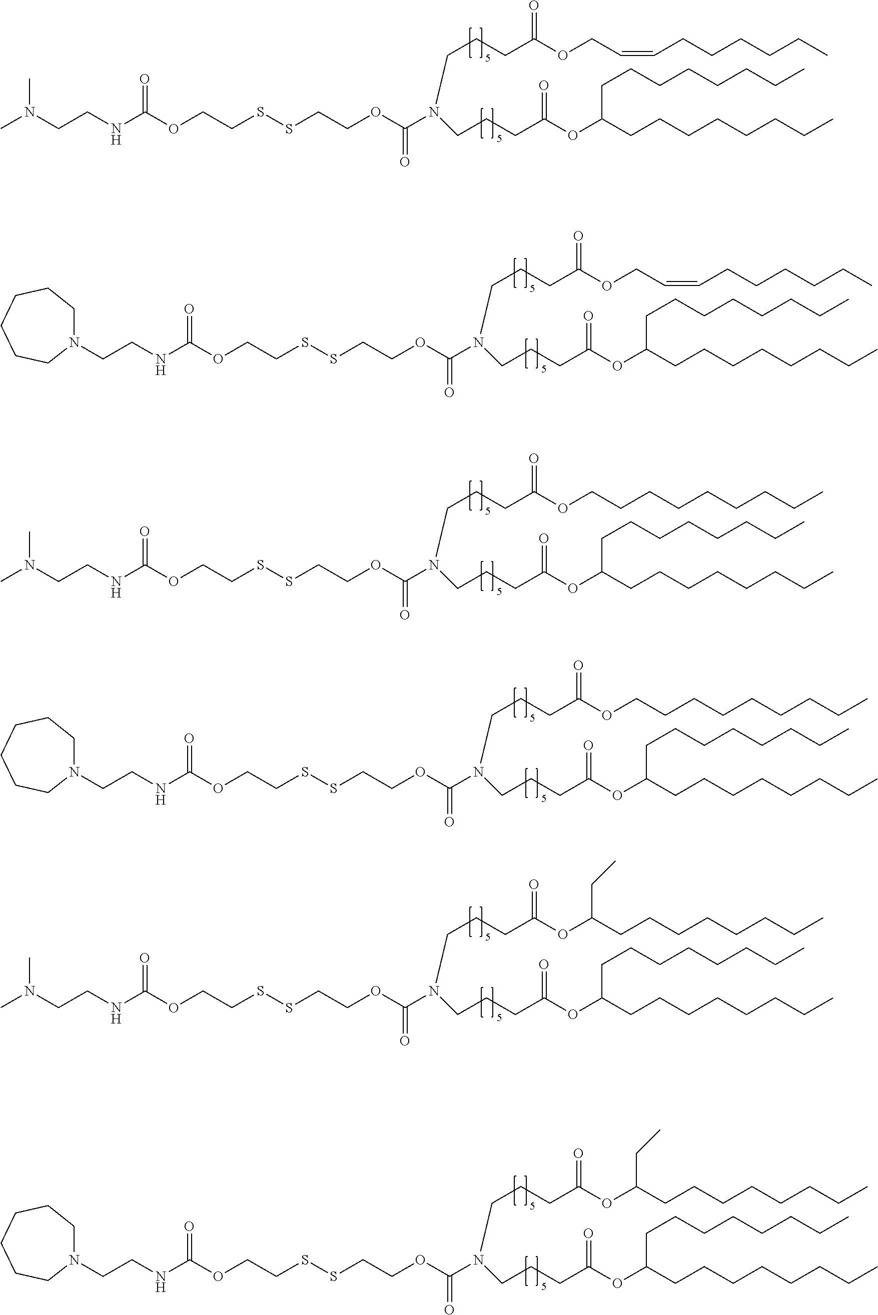

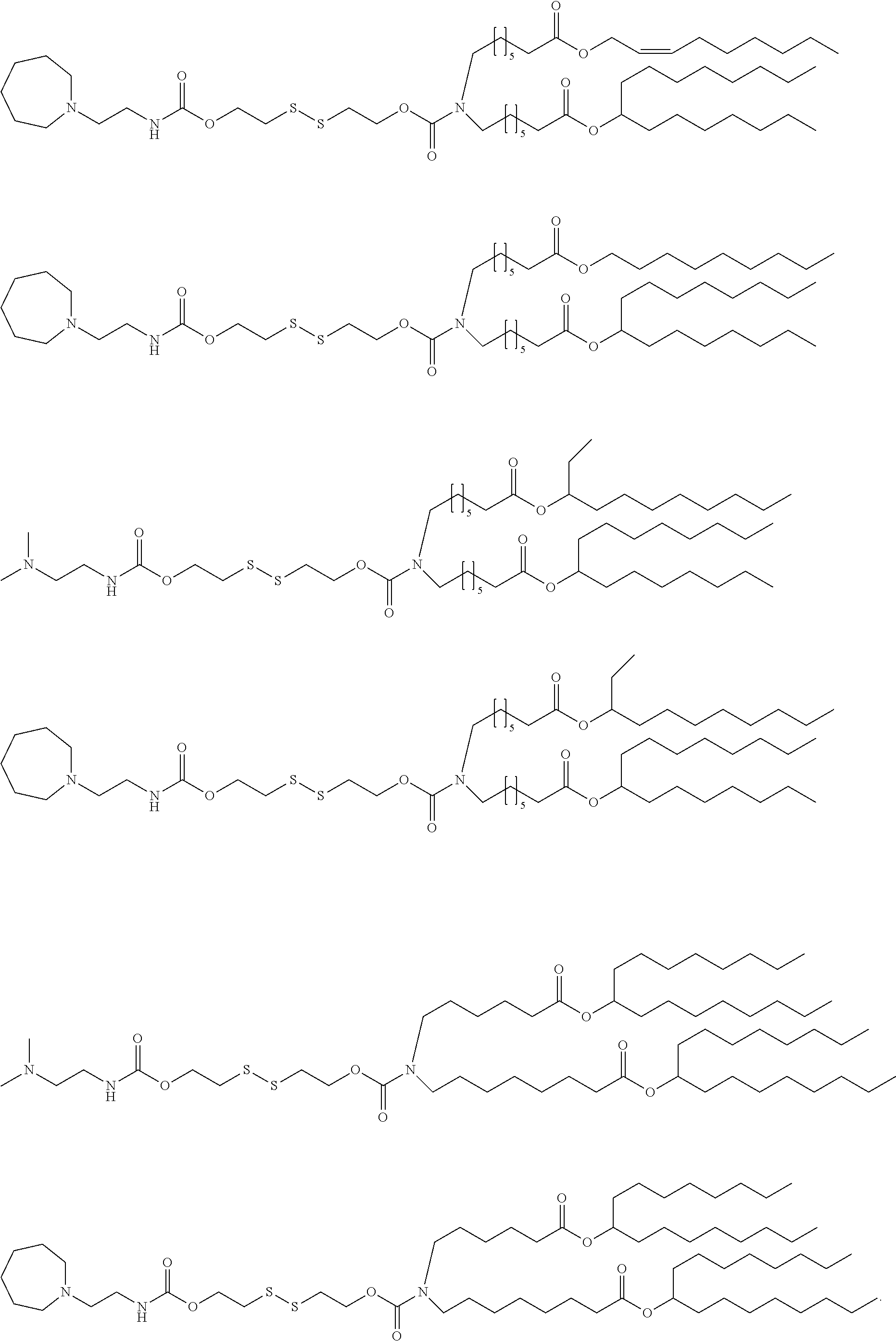

6. An ionizable lipid as defined in anyone of claim 1 or 2; wherein the total number of C atoms in R₁ and R₂ together is at least 14.

7. An ionizable lipid as defined in anyone of claim 3 or 4; wherein the total number of C atoms in R₇ and R₇″ together is at least 12.

8. An ionizable lipid as defined in anyone of claims 1 to 3 or 5; wherein m and n are the same, being an integer selected from 1, 2, 3 and 4; preferably 2.

9. An ionizable lipid as defined in anyone of claims 1 to 3 or 5; wherein R₅ is —NH—.

10. A lipid nanoparticle or lipid nanoparticle composition comprising an ionizable lipid as defined in anyone of claims 1 to 9.

11. The lipid nanoparticle or lipid nanoparticle composition according to claim 10, further comprising a phospholipid, a sterol and/or a PEG lipid.

12. The lipid nanoparticle or lipid nanoparticle composition according to anyone of claims 10 to 11, further comprising an active agent, in particular a nucleic acid, preferably mRNA.

13. Use of an ionizable lipid as defined in anyone of claims 1 to 9 in the manufacture of a lipid nanoparticle or lipid nanoparticle composition.

14. A pharmaceutical composition comprising a lipid nanoparticle or lipid nanoparticle composition as defined in anyone of claims 10 to 12, and a pharmaceutically acceptable agent.

15. A pharmaceutical composition as defined in claim 14 for use in medicine.
